(19) United States
(12) Patent Application Publication
(10) Pub. No.: US 2026/0290207 A1
(43) Pub. Date: Sep. 24, 2026

(54) MOUTH-THROAT MODELS FOR THE ASSESSMENT OF REGIONAL DEPOSITION OF INHALED PHARMACEUTICAL AEROSOLS

(71) Applicant: Virginia Commonwealth University, Richmond, VA (US)

(72) Inventors: Laleh Golshahi, Glen Allen, VA (US); John V. Wilkins, JR., Richmond, VA (US)

(21) Appl. No.: 19/555,594

(22) Filed: Mar. 3, 2026

Related U.S. Application Data
(60) Provisional application No. 63/765,913, filed on Mar. 3, 2025.

(51) Int. Cl. G09B 23/34; A61M 15/00; B33Y 50/00; B33Y 80/00

(52) U.S. Cl. CPC G09B 23/34; A61M 15/00; B33Y 50/00; B33Y 80/00; A61M 2205/70

(57) ABSTRACT

Airway models and sets of airway models capture anatomical morphologies of mouth-throat geometries. The models capture intersubject variability. In addition, a method of predicting deposition of a nebulized hygroscopic aerosol in at least one pediatric oral airway involves determining or receiving one or more airway geometries from the one or more medical images including at least one characteristic dimension of an oral airway and an airway volume; determining or receiving one or more delivery conditions for the nebulized hygroscopic aerosol; calculating multiple nondimensional parameters such as Tidal Volume/airway volume ratio (TV/V), Stokes (Stk) number, Reynolds (Re) number, and Schmidt (Sc) number; and determining deposition in the oral airway using the TV/V, Stk, Re, and Sc numbers.

### CROSS-REFERENCE TO RELATED APPLICATIONS

This application claims the benefit of U.S. Provisional Patent Application No. 63/765,913, filed Mar. 3, 2025, the complete contents of which are herein incorporated by reference.

### FIELD OF THE INVENTION

Embodiments of the invention generally relate to anatomical models and, more specifically, mouth-throat models suited to testing and development of inhalers and methods of delivery of therapeutics to patient airways.

### BACKGROUND

Inhaled aerosols are widely used for respiratory therapeutics and represent a key exposure pathway for airborne pollutants, engineered particles, and emerging nicotine delivery systems. While decades of research have focused on adult nasal and pulmonary deposition, the oral airway (mouth-throat) serves as the primary deposition site for larger, high-inertia droplets commonly emitted by pressurized metered-dose inhalers (pMDIs), dry powder inhalers (DPIs), nebulizers, and electronic cigarettes (ENDS). Understanding deposition in this region is essential because aerosols retained in the oropharynx do not reach the lung and therefore reduce effective clinical dose, while simultaneously increasing local exposure and side-effects.

Children represent a particularly understudied population in aerosol science. Pediatric oral airways differ significantly from adult geometries, with smaller diameters, shorter airway lengths, and sharper oropharyngeal curvature, resulting in flow regimes that can amplify inertial impaction and increase variability in lung delivery. At the same time, pediatric breathing is often more transient and variable than adult breathing, characterized by steep increases in inhalation flow at the start of the breath and substantial breath variability in inspiratory flow patterns, in contrast to the steady, constant flows frequently used in in vitro testing. As a result, many existing deposition data and models, which are based on adult steady-flow conditions and idealized geometries, cannot be directly translated to pediatric patients. Moreover, intersubject variability among children (ages 6-14) is significant, reducing the applicability of generalized models to individual treatment planning, as current in vitro airway models and correlations do not capture these anatomical and physiological differences.

Computational fluid dynamics (CFD) models can simulate some aspects of aerosol transport, but without comprehensive experimental validation, their predictive value remains limited. Historically, deposition estimations have relied on anatomical scaling or semi-empirical relationships that do not generalize across physiologically relevant conditions.

Existing predictive models don't fully account for complex interplay of biological and physical factors. Specifically, previous deposition correlations relied on stable, non-hygroscopic particles, failing to account for the influence of hygroscopic particle dynamics. Aerosol droplets, particularly those generated by nebulizers (e.g., albuterol sulfate), are often hygroscopic; upon inhalation, they encounter the warm, saturated environment of the respiratory tract, causing them to rapidly change size through condensation or evaporation. This size-shifting effect strongly alters the droplet's aerodynamic diameter and, consequently, its regional deposition pattern.

Individual oral airway models of nine children 6-14 years old and nine older individuals (17-41 years old) were developed by Dr. Laleh Golshahi during her PhD with Dr. Warren Finlay in Mechanical Engineering at the University of Alberta in Edmonton, Alberta, Canada. More information on the models/geometries can be found in the four references below. The models were used to develop correlations to predict the total mouth-throat losses in the 18 geometries with stable jojoba oil aerosols as described in the four references:
1. Golshahi, L., Noga, M. L., Vehring, R., and Finlay, W. H. (2013) An In vitro study on the deposition of micrometer-sized particles in the extrathoracic airways of adults during tidal oral breathing. Annals of Biomedical Engineering. 41(5): 979-989. Journal Impact Factor: 3.47.
2. Golshahi, L., Vehring, R., Noga, M. L. and Finlay, W. H. (2013) In vitro deposition of micrometer-sized particles in the extrathoracic airways of children during tidal oral breathing. Journal of Aerosol Science. 57: 14-21. Journal Impact Factor: 2.28.
3. Golshahi, L. and Finlay, W. H. (2012) An idealized child throat that mimics average pediatric oropharyngeal deposition. Aerosol Research Letter*, Aerosol Science and Technology. 46 (5): i-iv. Journal Impact Factor: 2.43.
4. Golshahi, L., Noga, M. L., and Finlay, W. H. (2012) Deposition of inhaled micrometer-sized particles in oropharyngeal airway replicas of children at constant flow rates. Journal of Aerosol Science. 49: 21-31. Journal Impact Factor: 2.28.

Related art further includes Yang, M., Ruzycki, C., Verschuer, J., Katsifis, A., Eberl, S., Wong, K., Golshahi, L., Brannan J. D., Finlay, W. H., and Chan, H. K. (2017) Examining the ability of Empirical correlations to predict subject specific in vivo extrathoracic aerosol deposition during tidal breathing. Aerosol Science and Technology. 51(3):363-376. Journal Impact Factor: 2.43.

### SUMMARY

Some exemplary embodiments are based on multiple adult and/or pediatric mouth-throat models. Some embodiments comprise a methodology to acquire medical images during controlled air inhalation. Each participant may be administered a controlled air administration (of, e.g., 12 Liters/min) through, e.g., an AKITA nebulizer during imaging. The value of 12 Liters/min is one non-limiting example of a particular controlled flow rate which may be selected to be reasonably close, but slightly and safely higher than typical inspiratory flow measured by spirometry for school-aged children 6-14 years old. There are correlations between height, weight, body surface area, and lung capacity metrics. Therefore exemplary methods of this disclosure may include determining and subsequently using a flow rate based on a subject's height and weight to estimate a safe flow slightly exceeding typical minute ventilation to prevent any unintended dilution of administered air by taking extra air from the room in a varying manner due to breathing flow exceeding the administered controlled flow. The images are then processed and models 3D printed based on the processed images. Such exemplary models may then be used in various experiments, and the resulting data is useful for making total mouth-throat deposition correlations for adults and/or children.

Some embodiments segment one or more models to smaller regions of interest to enable exploration of regional deposition of pharmaceutical aerosols such as nebulized solutions, suspensions, pressurized metered dose inhaler (pMDI) aerosols, and dry powder inhaler (DPI) aerosols. In this disclosure, “aerosol” is generally meant to encompass both liquid droplet aerosols and dry powder aerosols. In either case, exemplary computational models in this disclosure are particularly well suited to predicting deposition for hygroscopic aerosols (aerosols which change size during delivery, e.g., from humidification in a carrier gas and/or from humidity of natural airways).

Some embodiments provide new correlations and predictive models for aerosol deposition in airways including pediatric airways. Some correlations and predictive models are for non-volatile aerosol for air and/or helium-oxygen (heliox) under heated and humidified conditions. Some correlations and predictive models are for volatile aerosol for air and/or heliox carrier gases.

Embodiments include both tangible models and computational models usable to model aerosol deposition in airways. Results from use of their type of model may be used to determine one or more optimal delivery conditions for a particular therapeutic to be delivered, a particular oral delivery device to be used, and/or a particular subject to be administered a therapeutic.

In some embodiments, the following approach is exemplary: 1) Measuring the dimensions of the mouth-throat models in detail, and 2) Measuring deposition of nebulized solutions, pMDIs, and DPIs., The outcome is the knowledge of how the dimensions in combination with the positioning of the device leads to a certain patterns of regional deposition or targeting for various medical applications and/or to avoid side effects particularly with some asthma medications, which for instance potentially can adversely affect the vocal chords.

Based on the acquired medical imaging, multiple (e.g., three or more) geometries are developed which entail the anatomical morphology of the mouth-throat geometries and represent, e.g., the low, mean, and high deposition in adults and children. Some embodiments expand the number of geometries to more than three. Embodiments may collect more medical DICOM images, e.g. CT and/or MRI, to have a more detailed database of anatomical dimensions with the consideration of intersubject variability.

In some embodiments, only one specific type of mouthpiece may be used. Other embodiments may be configured based on information on how multiple different mouthpieces of inhalers impact the geometry of the mouth-throat.

Exemplary models may be used for testing of depositional characteristics of pharmaceutical aerosols from e.g. nebulizers, pMDIs, and DPIs. For instance, some methods of use may comprise measuring total drug mass and size of aerosolized drug solution leaving the mouth-throat model.

Some embodiments comprise one or more models that are sectioned to anatomical regions. These sectioned models may be used, for example, to test regional deposition of pharmaceutical aerosols and measure relationships between anatomical dimensions and regional dose. Exemplary models may be used to test deposition of aerosols and measure relationships between anatomical dimensions, physicochemical properties of device output e.g. mass concentration of solid or liquid phase in carrying gas across the cross section of airway also referred to as cloud effect, particle size, velocity and density/porosity, e.g. PulmoSphere by Novartis is a proprietary technology for powder formulation with spray drying, which makes the powder particles porous to lower impaction due to momentum while maintaining a certain size that can reach a desired target.

Exemplary sectioned models are a valuable tool for efforts due to propellants in pMDIs being considered as GWP gases, and there is a whole new market to redevelop pMDIs. They also uniquely lend themselves to nebulizer and DPI combination product testing and development.

Exemplary embodiments may comprise a set (i.e., kit) of standardized mouth-throat models with anatomical features representing the range of drug delivery to different regions for pharma product testing and development applications. An exemplary kit may include, for example, at least three pediatric models (or exactly three pediatric models), at least three adult models (or exactly three adult models), or a combination of three (or more) pediatric models and three (or more) adult models. For each triplet representing a particular subject population (e.g., pediatric vs adult), it is advantageous that one model be selected as representative of an anatomy in which comparatively high deposition may be expected, another be selected as representative of an anatomy in which comparatively low deposition may be expected, and another be selected as representative of an anatomy in which comparatively intermediate deposition may be expected. Such triplet models may be selected from a relatively large sampling of available anatomies such as but not limited to at least 10, at least 20, at least 30, at least 40, or at least 50 available geometries per subject population.

According to some embodiments, the geometries of exemplary models are developed based on high resolution medical images (such as CTs) collected during controlled inhalation. Some exemplary models are unique for pediatrics group and capture anatomical features precisely for pediatric subjects. Some exemplary models capture intersubject variability as opposed to one size fit all.

Some embodiments are configured to be easy to assemble and mount on ACI and NGIs.

Exemplary models may be made of or comprise solvent resistant material such as but not limited to metal.

Some embodiments comprise a set of pediatric and adult individual mouth-throat models for testing combination pharmaceutical aerosol drug delivery products in terms of regional extrathoracic deposition.

Keywords related to exemplary embodiments may include but are not limited to: Extrathoracic, Cast, Larynx, Mouth-throat, Inlet, Aerosol, and Inhaled Pharmaceutical Compounds.

An exemplary embodiment comprises a set of low, mean, high (deposition) mouth-throat models in adults and/or children. The “kit” of adult and pediatric mouth throat models allow comparative studies to extend the label dose established for adults to children based on the corrected loss of drug in upper airways, leading to a different fraction of emitted dose reaching the lungs.

Some exemplary embodiments are methods of using one or more exemplary airway models. An exemplary method of use may comprise determining total mouth-throat deposition correlations for adults and/or children. An exemplary method of use may comprise measuring regional deposition of pharmaceutical aerosols such as from nebulized solutions, suspensions, pressurized metered dose inhalers (pMDIs), and dry powder inhalers (DPIs). An exemplary method of use may comprise measuring total drug mass and size of aerosolized drug solution leaving the mouth-throat model. An exemplary method of use may comprise testing regional deposition of pharmaceutical aerosols. An exemplary method of use may comprise correlating anatomical dimensions with regional dose delivery for a given combination drug-device output allowing “screening” for the optimum drug device product delivery with the right amount of dose to the right target.

Exemplary geometries may be developed based on, e.g., high resolution CTs collected during controlled inhalation. The controlled inhalation make take into account the consideration of lung function, but an exemplary controlled inhalation may be achieved with a positive pressure configured to exceed natural inhalation peak flow slightly to prevent unintended dilution due to entrained room air. Ideally this flow should match the intended aerosol delivery flow. Consideration for change in morphology of airways with airflow and shape of mouthpiece allows more precision in estimating losses of drug in mouth-throat region, which in turn affects the delivered dose. Such precision is particularly important for drugs with narrow therapeutic index for applications such as oncology or drug-resistant bacteria leading to sepsis needing precise amount of antibiotics or antiviral therapeutics.

Some exemplary models may be unique particularly for pediatric group. Exemplary models may capture anatomical features precisely during inhalation of a controlled amount of flow rate to ensure the airway cavity is kept patent. An exemplary set of models captures intersubject variability as opposed to one average size.

The sectioning of the geometries in some models provides distinct anatomical regions which are a valuable tool for efforts required due to propellants in pMDIs being considered as global warming potential (GWP) gases and the market to re-develop pMDIs with zero carbon alternatives (currently mainly in Europe). They also uniquely lend themselves to nebulizer and DPI combination product testing and development.

Exemplary embodiments may be configured to be easy to assemble and mount on common pharmaceutical aerosol sizing equipment such as Anderson Cascade Impactor (ACI) and Next Generation Impactors (NGIs) or innovative lung models with more anatomical precision. They can act as an “inlet” to any representation of lung including microphysiological systems needing the knowledge of incoming lung dose.

Exemplary embodiments may be made of solvent resistant material such as metal, but manufacturing in metal while maintaining the anatomical accuracy is complex.

Embodiments include a set of mouth-throat geometries. As a non-limiting example, an exemplary set includes or consists of low, mean, high mouth-throat models that may partially be selected based on the methods similar to some of Dr. Golshahi's publications in other areas such as nasal drug delivery.

Exemplary models may be used to test transient (volatile, changing in size with time) pharmaceutical aerosols such as aerosols produced by nebulizers, pressurized metered dose inhalers (pMDIs), and dry powder inhalers (DPIs), which go through temporal changes resulting from evaporation and condensation. This may include recreational drug use situations such as but not limited to vaping devices (e.g., vaping pens). It is important to have a true characterization of lung delivery and inhalation exposure from vaping outputs—including nicotine, non-nicotine formulations, cannabinoids, and emerging controlled substances—to accurately assess their respiratory impact and associated health risks. Exemplary embodiments of this disclosure, including tangible physical models and correlative computational models, are able to provide data in these contexts without necessitating actual clinical trials with living human subjects.

Though this disclosure frequently refers to modeling deposition during inhalation, exemplary embodiments may also be employed for modeling deposition during exhalation and inhalation/exhalation cycles. For instance, the models of this disclosure may be used to determine the concentration of alcohol in a single exhalation based on the quantified amount of alcohol emerging from the exhaled breath to develop more non-invasive but precise breath analyzer (i.e., breathalyzer). This exemplary use is not just limited to alcohol and can be extended to other law enforcement applications on other controlled substances such as cannabinoids.

### BRIEF DESCRIPTION OF THE DRAWINGS

FIG. 1 is a pictorial flowchart for producing an exemplary airway model.

FIG. 2 shows multiple mouth-throat models, the set of which captures intersubject variability in mouth-throat of nine individuals. FIG. 2 also shows exemplary sectioning placement for exemplary models.

FIG. 3 shows two sets of mouth-throat (MT) airway models for testing pharmaceutical aerosol drug delivery products in terms of regional extrathoracic deposition.

FIG. 4 illustrates in vitro testing of an aerosol using an exemplary oral airway model.

FIG. 5 is a flowchart of an exemplary method of using one or more airway models.

FIG. 6 is a flowchart of another exemplary method of using one or more airway models.

FIG. 7 is a flowchart of an exemplary method of predicting deposition of a nebulized hygroscopic aerosol in at least one pediatric oral airway.

FIG. 8 is a flowchart of an exemplary method of medical treatment of a subject.

FIG. 9. Experimental Setups for Aerosol Measurements A) Aerosol deposition setup: Aerosol deposition was collected on the Pulmoguard filter and measured using high performance liquid chromatography (HPLC). B) Aerosol Sizing Flow chart: Aerosol sizing was measured using an optical particle sizer (OPS), an additional supplemental flow of 1 LPM, which was controlled using a mass flow controller, was added to system.

FIG. 10. Recovered mass at the filter (“Lung Dose”) under Air and Heliox conditions. A) Lung dose in micrograms (μg) for Air; B) Lung dose as percentage for Air; C) Lung dose in micrograms (μg) for Heliox; D) Lung dose as percentage for Heliox. Each measurement represents the mean of three replicates (n=3), with error bars indicating standard deviation.

FIG. 11. Percentage of nebulized dose recovered at the filter and deposited in the model under Air and Heliox conditions. A) Percentage of Nebulized Dose Delivered to Filter for Air and B) Percentage of Nebulized Dose Delivered to Filter for Heliox. C) Percentage Deposited in the Model for Air and D) Percentage Deposited in the Model for Heliox. Each measurement represents the mean of three replicates (n=3), with error bars indicating standard deviation.

FIG. 12. Comparative analysis of aerosol delivery metrics for each carrier gas under benchtop (unheated) and environmental chamber (heated) conditions. A) Lung dose (μg). B) Lung dose as a percentage. C) Percentage of Nebulized Dose Delivered to the Filter. D) Aerosol deposition percentages within the airway models. Each measurement represents the mean of three replicates (n=3), with error bars indicating standard deviation.

FIG. 13. Scatter plots and corresponding linear regression models comparing predictive performance (R²) using: Volume by surface area V/Aₛ and the square root of volume over length √(V/L) across gas-specific and universal models. A) Air vs Universal. B) Heliox vs Universal. Each marker represents an individual data point under a specific gas condition, and regression lines reflect the best fit for each grouping. Dashed lines indicate gas-specific fits; solid lines represent models fit to pooled data. R² values are reported for each model to assess predictive accuracy.

FIG. 14A. Schematic image of the experimental setup for the aerosol deposition measurements used in Example 3.

FIG. 14B. Schematic image of the experimental setup for the aerosol sizing measurements used in Example 3.

FIG. 15A. Recovered filter deposition with air as carrier in Example 3.

FIG. 15B. Recovered filter deposition with heliox as carrier in Example 3.

FIG. 16. Comparison of the filter mass deposition between the benchtop (unheated) tests and the environmental chamber (heated) tests.

FIG. 17. Linear regression fit of the actual mass deposition measured using HPLC compared to the predicted mass deposition in Example 3.

### DETAILED DESCRIPTION

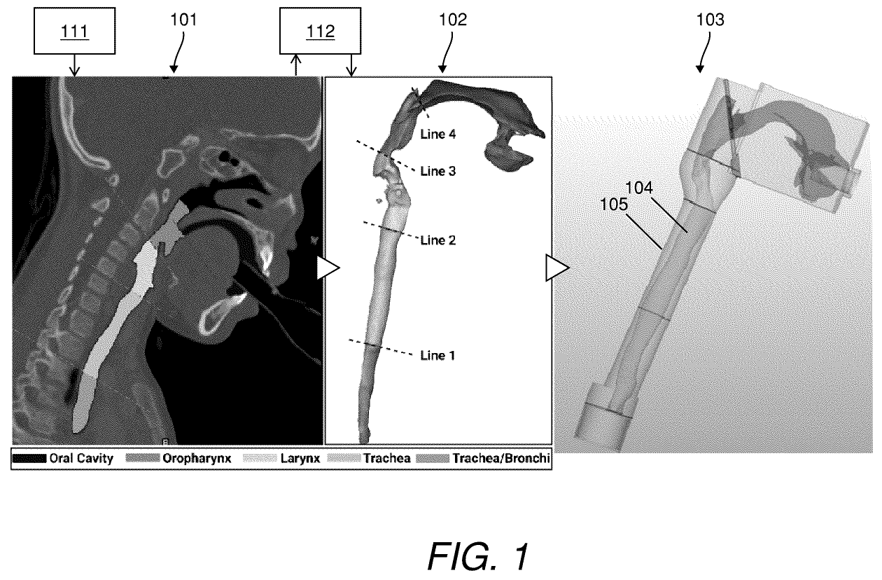

FIG. 1 is a pictorial flowchart for modeling according to an exemplary embodiment. One or more medical images, such as but not limited to the representative computed tomography (CT) image 101 in FIG. 1, of one or more subjects are first captured. The image 101 actually appearing in FIG. 1 is, in particular, a sagittal CT slice. The medical imaging includes patient airways, in particular, oral airways. Oral airways, as used in this description, refers to at least the mouth-throat (MT) region and may or may not also include the oropharynx, larynx, and trachea. An “oral airway” may or may not further include a start of the bronchi. Generally, the term “oral airway” may also be considered interchangeable with “extrathoracic airway” less the nasal cavity.

It is preferred that the medical images are high resolution such as but not limited to Digital Imaging and Communications in Medicine (DICOM) images. Exemplary DICOM images may be captured by computed tomography (CT) or magnetic resonance imaging (MRI), for example.

It is advantageous for the high resolution medical images to be collected during controlled inhalation of the subject. This helps reflect the most accurate anatomical geometry present when a subject is inhaling a therapeutic aerosol and ensures the airway cavity is kept patent.

From the medical imaging, one or more airway geometries (i.e., geometric features) from the one or more medical images 101 may be determined using one or more imaging devices 111. The medical imaging device(s) 111 may be, for example, an MRI machine, CT machine, or other medical imaging technology. Geometric features which are extracted from the image may include, for example, at least one characteristic dimension of an oral airway and a (total) airway volume for the airway in question. In FIG. 1, different shades have been overlaid on the CT image 101 to represent geometric feature extraction.

In some embodiments, the step of medical imaging to produce the necessary image(s) 101 may be advantageously efficient compared to existing techniques. With adult subjects, imaging may be conducted with gating. Gating in MRI, for example, is a synchronization technique used to reduce motion artifacts by timing image acquisition to specific phases of the motion cycle (e.g., of the respiratory cycle). Though gating may be used with adult subjects, the process can prove too long with pediatric subjects. Pediatric subjects may find it impossible to remain still for the time required by gating. To overcome this obstacle, exemplary imaging according to this disclosure may be conducted without gating and with positive air pressure during subject inhalation. An exemplary positive air pressure is a pressure (which may be steady state pressure) which is sufficiently high to prevent accidental inhalation by the subject of room air (e.g., room air through the nose while receiving the positive pressure through the mouth). The exact pressure will vary depending on the subject's inspirative capacity. Inspirative capacity generally trends with age such that young patients require comparatively lower pressure to older patients.

A further feature of some exemplary embodiments is that the medical image to produce image(s) 101 is conducted with the patient holding via his or her mouth an aerosol delivery mouthpiece. It is particularly advantageous, if possible, for the imaged subject to be holding via his or her mouth a mouthpiece substantially matching or identical to the mouthpiece the same or a different future subject will be using for actual aerosol administration, conditions of such administration being planned ahead of time based on the model 102 produced from the imaging 101. In effect, the imaging may be conducted with the subject inhaling as if an aerosol is being delivered though no aerosol is present during the imaging.

A three-dimensional (3D) oral airway model 102 is constructed from the medical images. The model 102 is segmented/sectioned into a plurality of parts each representing a different section of an airway. The processing of the image(s) 101 and production of the model 102 may be performed by one or more processors 112.

The plurality of parts from model segmentation include at least a first section modeling an upper part of an airway and a second section modeling a lower part of an airway. For instance, a first section may model the oral cavity and a second section may model the oropharynx and larynx. The oropharynx and larynx may be treated as a single section or may be separated into two adjacent sections. Further sections may be included, for example, a third section may model the upper trachea. The model 102 in FIG. 1 is illustrative of five segmented regions (oral cavity, oropharynx, larynx, trachea, and trachea/bronchi).

The model 102, which may be a digital model, is reproduced as a physical/tangible 3D oral airway model 103. The model 103 may be used for in vitro depositional studies, for example. The model 103 is advantageously suited for regional deposition analysis. That is to say, the model 103 may be used not only to assess total extrathoracic airway deposition during administration of an aerosol but also deposition within distinguishable key anatomical regions of the extrathoracic airway. FIG. 1 shows section lines (here, Line 1 to Line 4) used for defining the boundaries of the regional deposition analysis (e.g., sectional rinsing). The 3D oral airway model 103 may also be referred to as a 3D oral airway replica. The anatomical geometry defines the internal space 104 of the model 103, the surrounding walls 105 of which may be made by a 3D printing or casting process, for example. The model 103 may be made of a plastic (e.g., a thermoplastic) or metal, for example. For some end use purposes, the surfaces of the airway within the model 103 may be coated with one or more coatings.

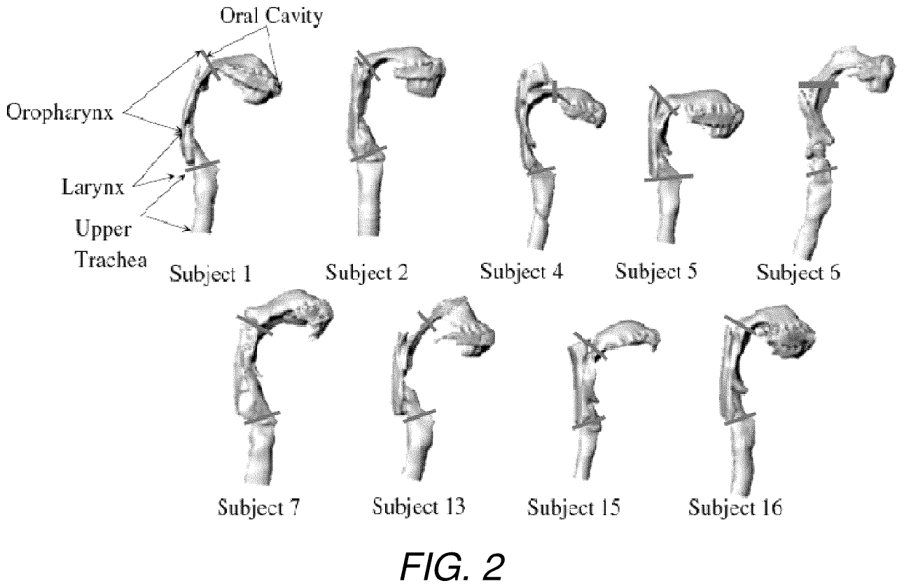

Some embodiments comprise a plurality of airway models that capture intersubject variability. By way of illustration, FIG. 2 shows nine different models, all of which have boundaries indicated to provide a three way segmentation. Each model in FIG. 2 has a first boundary separating the oral cavity from the oropharynx and a second boundary separating the larynx from the trachea. As FIG. 2 illustrates, the anatomy-replicating geometry of each model varies from one model to another, therefore the placement of the aforementioned boundary lines also varies from one model to another. As mentioned above in connection with FIG. 1, the sectioning enables the capture of regional deposition information when the models are used for assessing deposition of aerosols in subjects. The geometries of exemplary models may be sectioned to anatomical regions relevant to local drug delivery similar to what is shown in FIG. 2 (but not limited to the regions shown).

Exemplary sectioned models may be used to test regional deposition of pharmaceutical aerosols and develop a set of standard mouth-throat models with detailed anatomical features intact for pharma product testing and development. Exemplary anatomical mouth-throat geometries and exemplary models embodying such geometries of adults and children may be used for combination product development and testing.

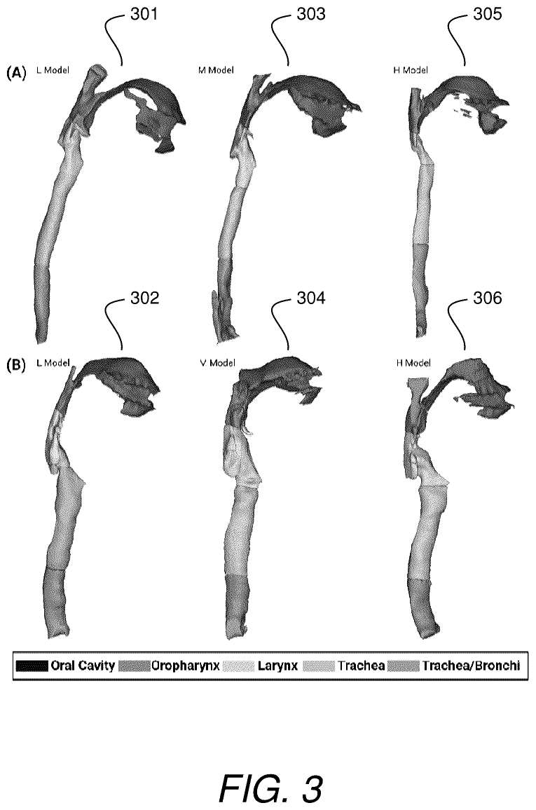

FIG. 3 shows two sets of mouth-throat (MT) airway models for testing pharmaceutical aerosol drug delivery products in terms of regional extrathoracic deposition. The first set comprises or consists of models 301, 303, and 305, all of which correspond with respective pediatric subjects. The second set comprises or consists of models 302, 304, and 306, all of which correspond with respective adult subjects. Each of the models 301-306 may be reproduced by, for example, the approach described above in connection with FIG. 1. Sets which entail at least three models or exactly three models are advantageous for testing aerosols. The three models are selected to respectively represent a low deposition airway, intermediate deposition airway, and high deposition airway, respectively. In FIG. 3, models 301 and 302 are low deposition airway models for children and adults, respectively. Models 303 and 304 are intermediate deposition airway models for children and adults, respectively. Models 305 and 306 are high deposition airway models for children and adults, respectively. The classification of any model as a low, intermediate, or high deposition may be made by testing an initially large set of models (e.g., at least 9, 10, 15, 20, 25, or 50 models, for example) representative of a particular subject group, e.g., just children or just adults, and testing the group with delivery of a preselected aerosol using preselected delivery conditions. From the results three models may be selected which represent empirical low, intermediate, and high deposition rates from within the group.

As was the case in FIG. 1, the models 301-306 are segmented into five different regions: Oral Cavity, Oropharynx, Larynx, Trachea, Trachea/Bronchi.

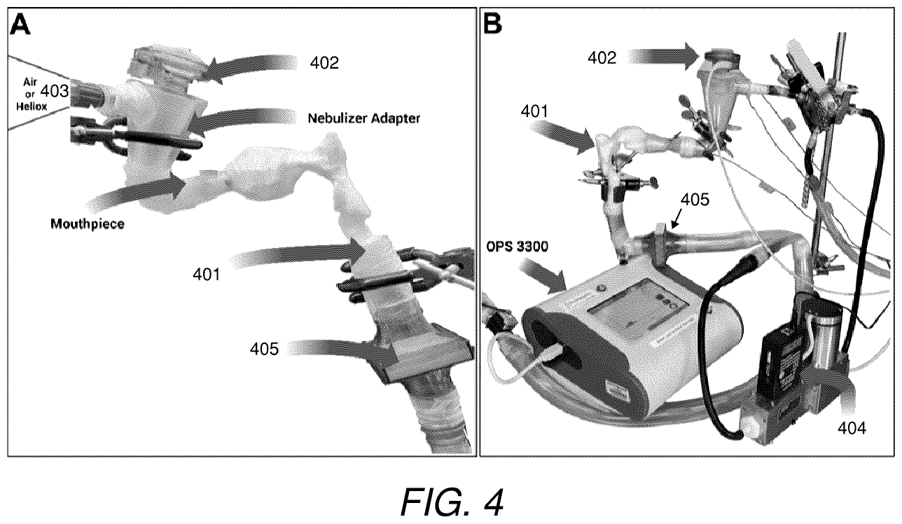

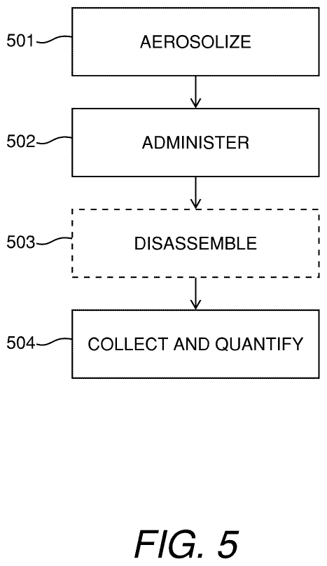

FIG. 4 illustrates an in vitro testing setup of an aerosol using an exemplary oral airway model 401. FIG. 5 is a flowchart of exemplary method of testing an aerosol and/or aerosol delivery device using an oral airway model. An aerosol generating device such as a nebulizer (e.g., a vibrating mesh nebulizer 402) aerosolizes (block 501) a therapeutic aqueous solution. The aerosol is combined with a carrier gas 403 such as air or heliox. The carrier gas 403 may be humidified and heated to replicate physiological humidity and temperature. The aerosol, along with the carrier gas if applicable, is administered (block 502) to the oral airway model 401. A flow regulator or mass flow controller 404 may be arranged upstream or downstream of the model 401 to simulate either steady state inhalation or a particular breathing pattern which may have variations in flow rate during a single administration of aerosol. The flow regulator/controller may be configured as a breathing simulator or used in combination with a breathing simulator. Breathing simulators are commercially available from, for example, Ingmar Medical, USA. The breathing simulator may be configured to account for a particular age group, e.g., children vs adults, the flow pattern to which the model is subjected.

A low-resistance filter 405 is arranged at the model outlet and is configured to collect any aerosol particles which do not deposit on surfaces inside the model. Regional and total deposition will be quantified via sectional rinsing and fluorescence analysis, complemented by UV imaging for spatial mapping. After the administration (block 502) of the aerosol is complete, any aerosol particles collected by the outlet filter are recovered and quantified (block 504), e.g., by high-performance liquid chromatography. Total deposition within the model may be determined by the difference of the amount of aerosol administered minus the amount of aerosol recovered at the outlet filter. Some exemplary models are disassembled (block 503), in which case the separate parts/sections of the model are separated from one another. Any aerosol depositions on each respective separate part/section of the model may then be recovered and quantified (e.g., by high-performance liquid chromatography), for region specific depositional data. The amount of aerosol recovered from the model outlet filter may be treated as the dose delivered to the lungs. This procedure may be conducted in an environmental chamber capable of controlling both temperature and humidity to eliminate any effect of cooling of the aerosol within the oral airway model.

The method of FIG. 5 may be repeated one or more times with different delivery conditions (e.g., one or more of: different therapeutic/medicament, different mode of aerosolization, different carrier, different dose, different flow rate, different temperature, different humidity, etc.) from the preceding test to provide a comparison of how any one or more these variables alters deposition within the model.

The method of FIG. 5 may furthermore be repeated for multiple different oral airway models yet with the same delivery conditions so establish deposition data which varies in dependence on anatomical differences among the multiple different oral airway models.

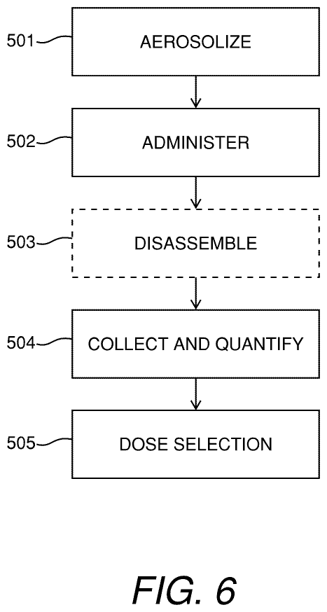

The determined deposition in the oral airway model or respective parts thereof may be the useful output of an exemplary method like that of FIG. 5. Determined deposition may be expressed as a fraction or percentage of dose administered or else may be expressed as actual quantity of the aerosol or the therapeutic/medicament within the aerosol (e.g., mg deposited). FIG. 6 is a flowchart which takes the deposition result(s) a step further. The results of the in vitro test determined at block 504 may in turn be used at block 505 to select/determine a dose to administer in vivo for an actual living subject/patient.

Some embodiments include predictive models, also called predictive computational models or predictive correlations in this disclosure, which give in silico results for deposition estimates. An exemplary computational model may be used to predict deposition of hygroscopic aerosol droplets in pediatric airways, for example. Exemplary predictive correlations incorporating tidal volume (TV) and a plurality of dimensionless numbers, in particular, Stokes, Reynolds, and Schmidt numbers. The predictive correlations also use at least one characteristic dimension and an airway volume of the oral airway of the oral airway being tested/evaluated. The volume-to-surface area ratio (V/Aₛ) is a preferred characteristic dimension, though some embodiments may use the square root of the volume-to-length ratio (√V/L).

Predictive models may weight different factors differently depending on whether the airway which is modeled is intended to be subject specific or generalized to a subject population. In the individual, gas specific models (specifically using V/Aₛ), the deposition pattern differs by carrier gas. For Air, TV/V is the dominant factor, followed by Re, Stk, and Sc contributing the least. For Heliox, Stk was the leading factor, followed by TV/V, Re, and Sc again the least. Taken together, these results indicate that although Re is most influential when fits are combined, the relative importance of other parameters is gas-dependent: TV/V drives deposition in Air, whereas Stk is most influential in Heliox, with Sc consistently the least important across conditions. Exemplary methods may weight these parameters accordingly. For example, an exemplary predictive model for an aerosol administration of a specific individual with air as the carrier gas may apply weights (e.g., different coefficients in a weighted sum) which give, for example, TV/V the largest weight, Re the next largest weight, Stk the third largest weight, and Sc the fourth largest weight.

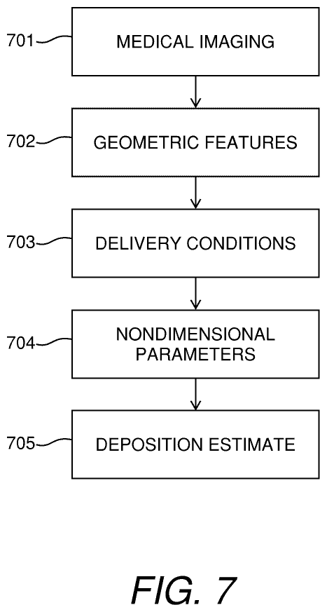

FIG. 7 is a flowchart summarizing an exemplary method of predicting deposition of a nebulized hygroscopic aerosol (such as but not necessarily limited to a nebulized hygroscopic aerosol) in at least one oral airway (which may be but is not necessarily a pediatric oral airway). Block 701 is capturing or receiving one or more medical images of at least one subject (e.g., pediatric subject). Block 702 is determining or receiving one or more airway geometries from the one or more medical images including at least one characteristic dimension of an oral airway and an airway volume. Block 703 is determining or receiving one or more delivery conditions for the aerosol. Block 704 is calculating Tidal Volume/airway volume ratio (TV/V), Stokes (Stk) number, Reynolds (Re) number, and Schmidt (Sc) number based on the one or more delivery conditions for the aerosol and one or more geometries. Block 705 is determining deposition in the oral airway using the TV/V, Stk, Re, and Sc numbers. Deposition may be estimated for an entire airway or for each of multiple distinct regions/segments of the airway.

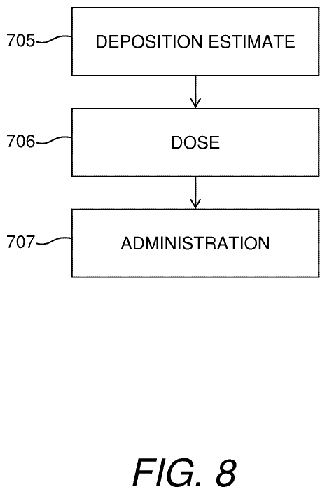

In some embodiments, the deposition estimate may be compared against deposition estimate(s) from a corresponding physical oral airway model. That is to say, in vitro and in silico results may be compared for administration of an aerosol to an oral airway modeled both physically and digitally from the same underlying medical image data. FIG. 8 shows a further use of the results of the method of FIG. 7, namely a dose 706 may be determined for an actual living subject from the deposition estimate 705, and the determined dose 706 may then be administered at block 707 to the living subject with a high degree of confidence as to the deposition behavior. Exemplary methods include variations of the methods depicted by FIGS. 7 and 8, including but not limited to specific applications of dose determinations and other applications of the models including those discussed in the Summary section of this disclosure.

The nondimensional parameters determined at block 704 of FIG. 7 and employed in the deposition estimate of block 705 may vary depending on qualities of the aerosol, delivery device, or combination aerosol-device of respective use contexts. For example, a hygroscopic aerosol with micron scale particle sizes, the parameters determined and employed may include the following: Tidal Volume/airway volume ratio (TV/V), Stokes (Stk) number, Reynolds (Re) number, and Schmidt (Sc) number. As an alternative example, when modeling a hygroscopic aerosol with much smaller particle size, e.g. nanoparticles, the deposition estimate may be further determined on the basis of Peclet number (Pe) which accounts for diffusive transport that plays a more significant role as aerosol particle size is reduced.

### EXAMPLES

### Example 1. Model Segmentation and Characterization

Three oral airway replicas were developed using anonymized in vivo Computed Tomography (CT) images of healthy subjects with no airway disease, deformity, or congestion. These replicas represent adults (ages 32, 32 and 41) and children (ages 6, 10, and 12), corresponding to low, medium, and high deposition patterns. The exclusion criteria to select the healthy subjects were opacification, mass, trauma, evidence of prior surgery, and presence of nasal or oral tube. Mimics software package (Materialise, Belgium) was used to segment the CT images based on the threshold level of air and develop the 3D mouth-throat airway models. Since the aim of this Example was to evaluate regional aerosol deposition fraction in oral airway models, the replicas were built in multiple pieces. The airway models were exported as STL file from the Mimics software and divided into several pieces with user defined cutting planes in 3-matic software (Materialise, Belgium). All these cutting planes are shown in FIG. 1, and the final representative models for Low, Medium, and High Deposition for adults and pediatrics are shown in FIG. 3.

The advantage of sectioned models is quantitative measurement of drug dosage in each piece, which allows in vitro evaluation of regional targeting. The end of each 3D airway model, exported from Mimics, was extruded 2-3 centimeters beyond the larynx to minimize the jet effects of larynx at the sampling point and for easier connection to the downstream tubing. All of the pieces were attached with a fair number of pins designed with a diameter of 2 mm and height of 4 mm, which kept each piece perfectly aligned and connected to the other pieces.

The 3D computer processed models are built using Stereolithography (SLA) by 3D Systems® Quickparts (3D Systems, Rock Hill, SC). The material is Accura ClearVue, which is a high clarity rigid plastic. The printing resolution was standard (0.004″ layer thickness) for all pieces. A quick finish process was performed on all printed pieces leading to a smooth surface of the replicas. The oral cavity piece of each replica is to be built of a flexible material (Formlabs Elastic 50A). Translucent material was chosen in order to further maneuver and manipulate the insertion of drug delivery devices into the mouth. An example of a 3D oral airway replica is illustrated in FIG. 1.

To quantify the amount of regional oral deposition, each of the oral airway models were segmented into five regions: oral cavity, oropharynx, larynx, trachea, and trachea/bronchi. The geometric parameters of each piece including the volume (mm³) and surface area (mm²) of the airways were previously determined using the 3-matic software and are presented in Tables 1 and 2. These geometric parameters are provided for the purpose of comparing sub-regional deposition in oral airway models.

TABLE 1
Volume (mm³) and surface area (mm²) of the oral airway regions in each of the three
anatomical airway replicas of the 6-year-old, 10-year-old and 12-year-old,
representing low, medium and high deposition.

| | L Model | | M Model | | H Model | |
|---|---|---|---|---|---|---|
| | | Surface | | Surface | | Surface |
| | Volume | area | Volume | area | Volume | area |
| Regions | mm³ | mm² | mm³ | mm² | mm³ | mm² |
| Oral Cavity | 7627.68 | 5303.98 | 18772.16 | 7832.21 | 22290.91 | 8775.56 |
| Oropharynx | 3517.53 | 1928.55 | 5254.38 | 2099.83 | 4067.62 | 2012.91 |
| Larynx | 2227.90 | 1465.68 | 1755.42 | 1039.28 | 3982.59 | 2120.10 |
| Trachea | 2968.00 | 1493.82 | 3666.21 | 1858.82 | 3580.44 | 1628.48 |
| Trachea/Bronchi | 2521.69 | 1423.77 | 5620.14 | 2954.33 | 1092.61 | 611.20 |

TABLE 2
Volume (mm³) and surface area (mm²) of the oral airway regions in each of the three
anatomical airway replicas of the 30-year-old, 30-year-old and 41-year-old,
representing low, medium and high deposition.

| | L Model M | | M Model S | | H Model J | |
|---|---|---|---|---|---|---|
| | | Surface | | Surface | | Surface |
| | Volume | area | Volume | area | Volume | area |
| Regions | mm³ | mm₂ | mm³ | mm² | mm³ | mm² |
| Oral Cavity | 34643.68 | 11582.45 | 32545.62 | 11544.76 | 44715.82 | 11492.94 |
| Oropharynx | 2647.07 | 1610.43 | 7673.51 | 3121.81 | 9155.20 | 3039.40 |
| Larynx | 9438.92 | 3730.28 | 12646.95 | 4263.11 | 25282.07 | 6674.98 |
| Trachea | 20560.45 | 5985.25 | 24240.38 | 5752.35 | 26022.18 | 6011.48 |
| Trachea/Bronchi | 13182.01 | 3745.49 | 17877.46 | 4385.50 | 18908.36 | 4891.09 |

### Example 2. A Multivariate Predictive Model for Pediatric Drug Delivery

### Pediatric Oral Models

Nine oral airway replicas were previously developed from CT imaging [27], [37]. Each model was 3D printed in two halves using an Objet24 printer (Stratasys, Israel) with a (0.05) mm resolution. This high resolution ensured that the inside of each model was clean and rid of all support material. Each model was then sealed together using an epoxy resin and was tested for leaks by running compressed air through them while submerged in a water bath. A custom-designed adapter was used to introduce aerosol to the humidified carrier gas from the Vapotherm Precision Flow machines (Vapotherm, MN, USA). This adapter was printed using a clear plastic material (Accura ClearVue, 3DSystems, USA), with a (0.05) mm resolution.

### Aerosol Deposition Measurements

Realistic breathing patterns for the child models were generated using a breathing simulator (ASL5000, Ingmar Medical, USA). The simulated flow pattern was measured using a low resistance flow meter (SFM 3000, Sensirion, Switzerland). To collect the dose that bypassed the oral airways, a low resistance filter (Pulmoguard filter, Quest Medical, USA) was attached to the model's outlet in each test. Heated and humidified Air and Heliox, were delivered using their respective Vapotherm units. The aerosol was delivered to the models using a vibrating mesh nebulizer (Aeroneb Lab, Aerogen, Ireland). The model drug used was a 2 mg/mL albuterol sulfate solution, which is commonly used in asthma inhaler medications. Nebulization was controlled using a mass dose controller (DSITM Buxco, MN, USA), which was used to control the duty cycle (the ratio of nebulized time to total nebulization time), cycling every six seconds, and also in the total nebulization time.

Analysis of the filter deposition was accomplished using high-performance liquid chromatography (HPLC). The HPLC method used in this study is based on the one described in [47] with a Hypersil Gold™ PFP HPLC column (5 μm pore size, 3 mm diameter, 150 mm length) (Waters Corp. Milford, MA).

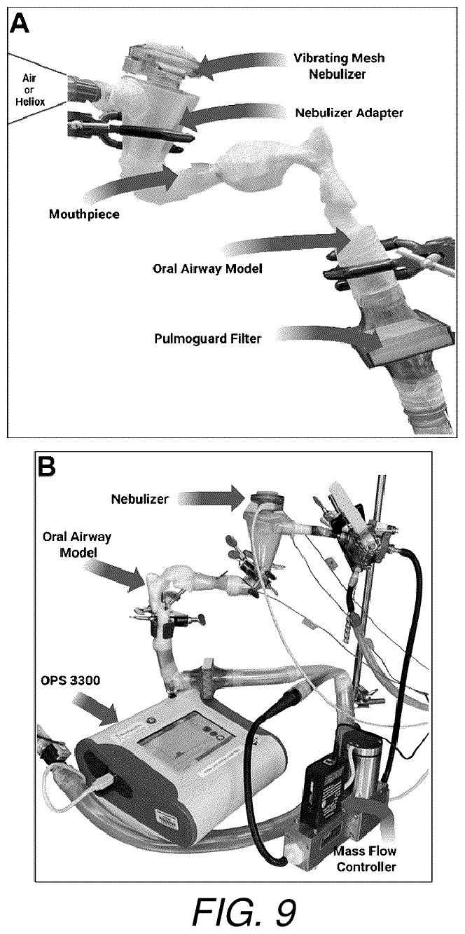

The experimental setup is shown in Panel A of FIG. 9. The oral models were connected to the breathing simulator, with specific breathing patterns detailed in Table 3. A Pulmoguard filter (Quest Medical, USA) was positioned at the model's outlet to quantify the undeposited aerosol. In a separate set of experiments, the filter was placed at the model's inlet to measure inhaled dose (the total drug entering the model).

TABLE 3
Model specific breathing parameters for assessing CO2 washout

| | | | | Approximate |
|---|---|---|---|---|
| Breathing | | RR | TV | Average Flow |
| Pattern | Description | [min−1] | [mL] | Rate [L/min] |
| BP1 | 5 year old | 23 | 174 | 8 |
| | (resting/sleeping)^{[13]} | | | |
| BP2 | Average of 43 | 20 | 385 | 15.4 |
| | patients 6-18 | | | |
| | years old^{[4]} | | | |
| BP3 | 15 year old | 20 | 490 | 19.4 |
| | (sitting awake)^{[13]} | | | |

The collected mass of the drug on the filters is presented in two ways: 1) as a percentage of nebulized drug mass, 2) as a percentage of the inhaled dose. Equations 1 and 2 describe the calculations for the mass collected on the filter and mass entering the model. model.

$${m}_{filter} = \frac{{C}_{sol} * 5 ml}{1000} \qquad ( 1 )$$

$${m}_{entered} = \frac{{m}_{nebulized}}{{m}_{inhaled}} * {C}_{sol} \qquad ( 2 )$$

where, Cₛₒₗ is the concentration of the solution measured by the HPLC, and 5 mL is the wash volume used during sample collection. Equation 3 calculates the total mass of drug on the filter (m_{filter}) for each model. Equation 4 estimates the mass of drug that actually entered the system (m_{entered}) based on the ratio of nebulized mass (m_{nebulized}) to inhaled mass (m_{inhaled}). The deposition percentage for each model was then determined by comparing the recovered filter mass to the inhaled dose for its corresponding breathing pattern.

### Aerosol Sizing Measurements

In this study, the Pulmoguard filter previously utilized for deposition measurements was replaced with an OPS3300 optical particle sizer (TSI, MN, USA) to enable direct particle size measurement, as shown in Panel B of FIG. 9. Standard-sized polystyrene particles were then delivered via a jet nebulizer, utilizing dry air and heliox as carrier gases. Further analysis confirmed good agreement between measured and expected particle sizes.

Aerosol sizing measurements were performed at the exit of the mouthpiece (prior to entering the model) and at each model outlet. The difference in these profiles indicates the fraction of the aerosol deposited within the model. To account for the flow rate of the OPS, an additional 1 LPM was supplied by the Vapotherm, so that the flow through the model remained consistent between deposition and sizing experiments. A mass flow controller (MCR-250SLPM, Alicat Scientific, USA) regulated this additional flow. Aerosol sizing was measured under the same breathing patterns applied to each model.

### Temperature Considerations

Since a temperature difference between the model (room temperature) and the carrier gas flow rate (37° C.) is to be expected, the temperature was measured at four locations: 1) at the carrier gas inlet where aerosol is introduced to the system, 2) within the mouthpiece immediately before the model inlet, 3) immediately following aerosol deposition filter, and 4) ambient temperature. This was done using four Type K thermocouples to allow us to evaluate the temperature drop across the system to determine the significance of cooling within the system.

Thermocouple locations are illustrated in FIG. 9 Panel B. Additionally, the average temperature between the model inlet (Point 2) and the filter outlet (Point 3) was used to determine carrier gas properties (density and viscosity) used for the evaluating non-dimensional correlations.

To determine if aerosol deposition differences occurs due to temperature differences between bench-top (room temperature) and body temperature (37° C.), models representing highest, intermediate, and lowest deposition rates were tested in an environmental chamber capable of controlling both temperature and humidity. The relative humidity inside the chamber was set to approximately 58%, matching the humidity recorded at the location where bench-top experiments were conducted and verified using a hygrometer. The chamber temperature was maintained at 37° C. to simulate normal body temperature. Deposition and sizing measurements followed the same experimental setups shown in FIG. 9.

### High Performance Liquid Chromatography (HPLC) Method

The HPLC method used in this Example was based on the method described for albuterol sulfate in R. R. Delvadia, P. W. Longest, and P. R. Byron, “In vitro tests for aerosol deposition. I: Scaling a physical model of the upper airways to predict drug deposition variation in normal humans.,” J. Aerosol Med. Pulm. Drug Deliv., vol. 25, no. 1, pp. 32-40, Feb. 2012. The injection volume was 100 μL, and a Hypersil Gold™ PFP HPLC column (Thermo Scientific, USA) was utilized. The system used was a Shimadzu LC system equipped with a UV-Vis detector (Shimadzu, Japan), with the UV detection wavelength set to 276 nm. The column and detector temperatures were set to 25° C. The flow rate was set to 0.75 mL/min, which resulted in a retention time of approximately 1.6 min. The HPLC method was evaluated for linearity using twelve standard concentrations, ranging from 0.50-165.6 μg/mL. Two stock solutions with concentrations of 165.6 and 98.0 μg/mL were used to prepare the standards by serial dilution. The calibration curve was checked for linearity using both groups of standards (R²≥0.999).

### Statistical and Regression Analysis

In this Example, the statistical analysis fulfills three main objectives: 1) showing how various independent variables influence deposition, 2) comparing nebulizer output across different gas and breathing pattern conditions, and 3) demonstrating a predictive model for deposition based on dimensionless parameters. To address the first objective, two full-factorial repeated-measures analyses of variance (ANOVAs) were performed using JMP® Pro 17.0.0 (SAS Institute Inc., USA). One ANOVA considered the effects of carrier gas (Air vs. Heliox) and breathing pattern (BP1, BP2, BP3), while the other focused on carrier gas (Air vs. Heliox) and heat condition (heated vs. unheated). By performing these separate ANOVAs, it was possible to isolate how breathing pattern and heating affect deposition, both individually and in combination with gas type.

In order to support the second objective (comparing nebulizer output) Student's t-tests examined potential differences in the mass of albuterol delivered under each gas type and each breathing pattern. This helped rule out the possibility that any observed differences in deposition surfaced simply from changes in total drug output.

The statistical significance level for all analyses was set at 0.05, with multiple comparisons further assessed through Tukey's Honestly Significant Difference (HSD) and Student's t-tests to pinpoint specific differences among conditions. In figures, a single asterisk (*) denotes a p-value less than 0.01, two asterisks (**) indicate a p-value less than 0.001, and three asterisks (***) represent a p-value less than 0.0001.

Finally, the third objective demonstrates the relationship between flow-related parameters (including geometry) and aerosol deposition. This Example presents a stepwise regression model incorporating dimensionless quantities known to influence particle transport under tidal breathing. Specifically, the predictors considered were the ratio of tidal volume to airway volume (TV/V), the Stokes number (Stk), the Reynolds number (Re), and the Schmidt number (Sc). Stepwise regression was conducted in JMP® Pro 17.0.0 (SAS Institute Inc., USA), with model selection driven by minimizing the corrected Akaike Information Criterion (AICc), which accounts for model complexity and sample size. Prior to regression, pairwise correlations among all candidate predictors were evaluated to identify multicollinearity. When strong correlations (Pearson's r>0.6) were found, the variable with greater physical interpretability or relevance to prior research was retained. Variable importance was quantified using JMP® Pro 17.0.0 total effect metric, accounting for both direct effects and interactions. This approach provided a comprehensive evaluation of each variable's contribution to deposition outcomes by accounting for both direct effects and interactions, rather than relying solely on individual statistical significance.

### Predictive Correlations

The aerosol deposition measurements obtained from the HPLC and the size measurements are used to determine the predicted deposition from the correlations. Since only the amount of aerosol deposited on the Pulmoguard filter was measured, the following method (Equation (3)) is utilized to determine the amount of aerosol deposited in the airways:

$${m}_{deposited} = {m}_{entered} - {m}_{filter} \qquad ( 3 )$$

where m_{deposited} is the mass deposited in the airway, m_{filter} is the mass of albuterol recovered from the filter and measured via HPLC, and m_{entered} is the estimated mass of drug that entered the system. Additionally, the deposition percentage was calculated with m_{deposited} and m_{entered}, as seen in Equation (4):

$${\%}_{deposited} = \frac{{m}_{deposited}}{{m}_{entered}} * 100 \qquad ( 4 )$$

Next, aerosol sizing measurements were used to refine the prediction of aerosol deposition in the oral airways. The particle size was determined across 16 bins, and for each individual experiment, the median diameter (D50) was utilized.

By incorporating these sizing measurements, the established non-dimensional correlation can be applied to predict the aerosol deposition in the oral airways using the Tidal Volume/Volume ratio (TV/V), Stokes (Stk), Reynolds (Re) and Schmidt (Sc) numbers, which are defined as:

$$Re = \frac{4 {ρ}_{gas} {\overline{Q}}_{inh}}{π {μ}_{gas} {d}_{c}} \qquad ( 5 )$$

$$Stk = \frac{2 {ρ}_{water} {d}_{a}^{2} {C}_{c} {\overline{Q}}_{inh}}{9 π {μ}_{gas} {d}_{c}^{3}} \qquad ( 6 )$$

$$Sc = \frac{{μ}_{gas}}{{ρ}_{gas} {D}_{gas}} \qquad ( 7 )$$

$$\frac{TV}{V} = \frac{Tidal Volume}{{V}_{model}} \qquad ( 8 )$$

where ρ_{gas} is the density of the carrier gas (Air at 33° C.: 1.139 kg/m³, 37° C.: 1.105 kg/m³; Heliox at 33° C.: 0.386 kg/m³, 37° C.: 0.377 kg/m³), Q̅ᵢₙₕ is the average flow rate during inhalation, μ_{gas} is the viscosity of the carrier gas (Air at 33° C.: 1.874 E-05 kg/m-s, 37° C.: 1.893 E-05 kg/m-s; Heliox at 33° C. 2.22 E-05 kg/m-s, 37° C.: 2.25 E-05 kg/m-s), D_{gas} is the mass diffusivity of water in the carrier gas (Air at 33° C.: 2.81 E-05 m²/s, 37° C.: 2.87 E-05 m²/s; Heliox at 33° C. 6.1206 E-05 m²/s, 37° C.: 6.259 E-05 m²/s), d_{c} is the characteristic dimension of the airway, ρ_{water} is the density of water (1000 kg/m³), dₐ is the aerodynamic diameter of the particle. Lastly, C_{c} is the Cunningham slip correction factor defined as:

$${C}_{C} = 1 + \frac{λ}{{d}_{p}} [ 2.34 + 1.05 exp ( - 0.39 \frac{{d}_{p}}{λ} ) ] \qquad ( 9 )$$

where λ is the mean free path of the carrier gas (Air: 72 nm; Heliox: 147 nm) and dₚ is particle geometric diameter.

### Aerosol Deposition

Aerosol deposition was quantified by measuring the mass of the drug captured on the filter placed either at the mouthpiece outlet (inhaled deposition) or at the model outlet, using HPLC analysis. To account for nebulizer output variability between replicates, the recovered drug mass was normalized relative to the emitted dose for each breathing pattern. This approach removed any differences with regards to the nebulized dose.

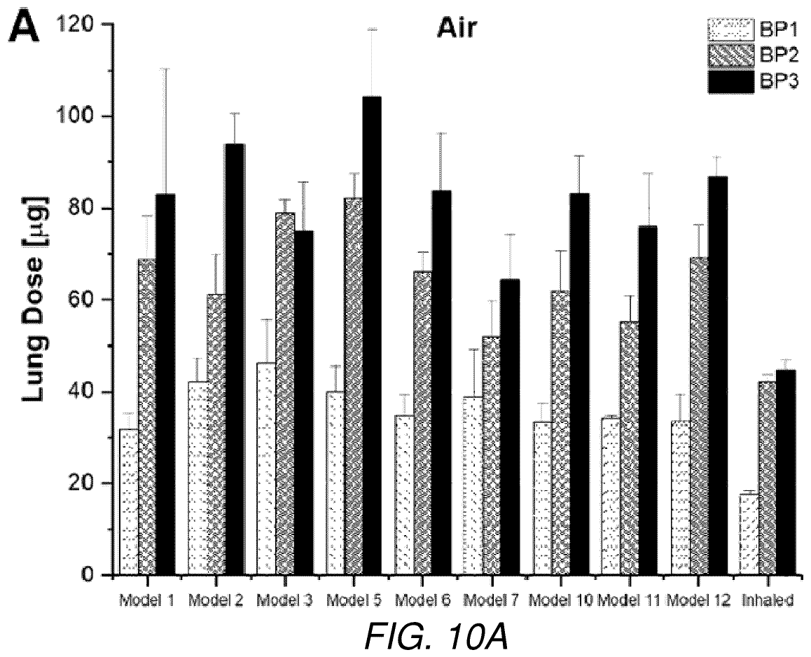

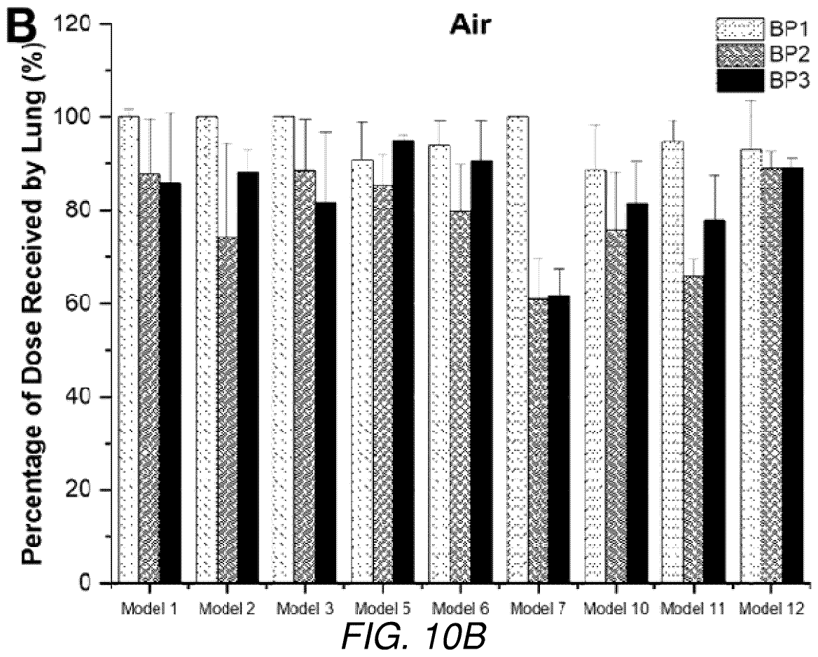

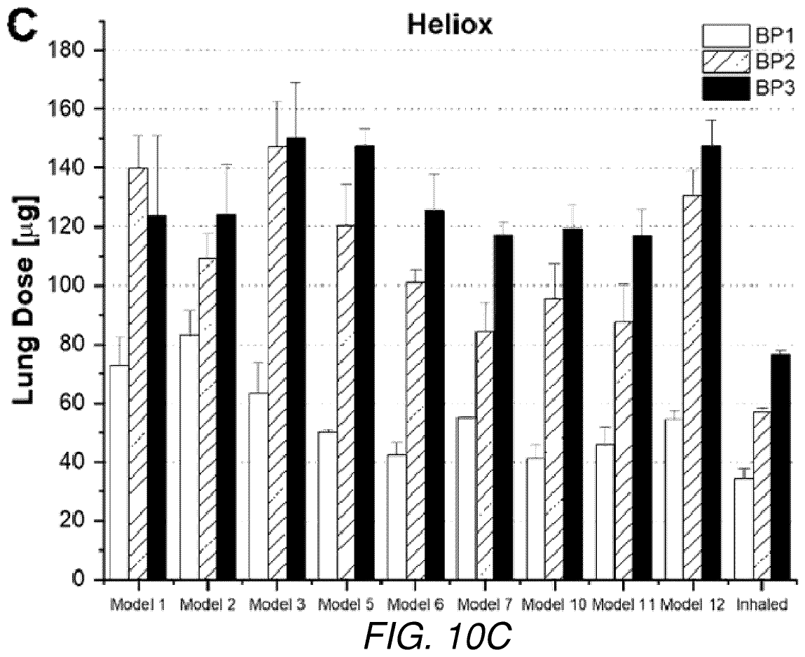

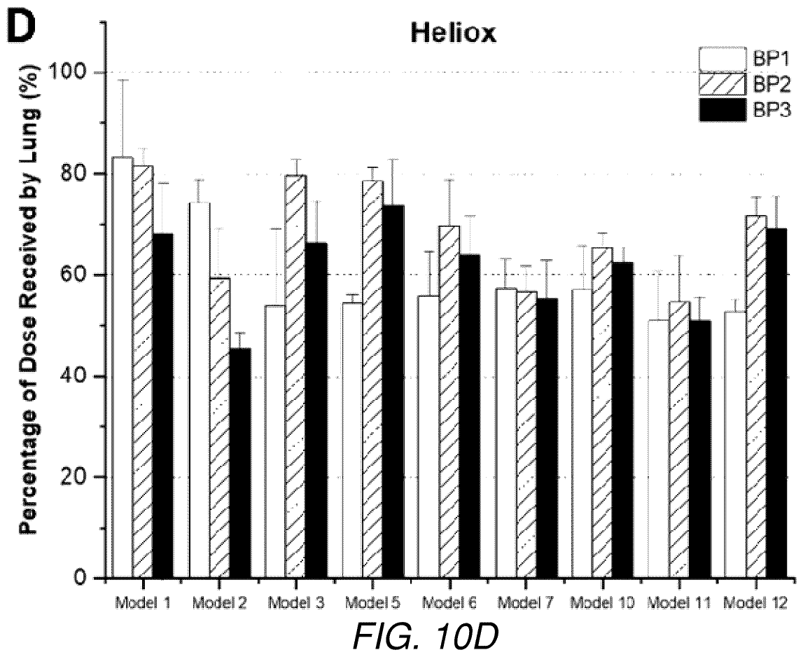

FIGS. 10A-10D present the recovered filter deposition, referred to as “Lung Dose”. FIGS. 10A and 10B show the lung dose in micrograms (μg). FIGS. 10C and 10D present the average percentage of lung dose relative to inhaled dose. To examine the effect of inhalation volume on aerosol deposition, three distinct breathing patterns which were previously detailed in Table 3, were tested for both carrier gases at a constant aerosol flow rate of 30 LPM. As the inhalation volume increased from BP1 to BP3, the recovered drug mass on the filter also increased. Flow rate measurements were recorded to account for any variations introduced by the Mouth-Throat models.

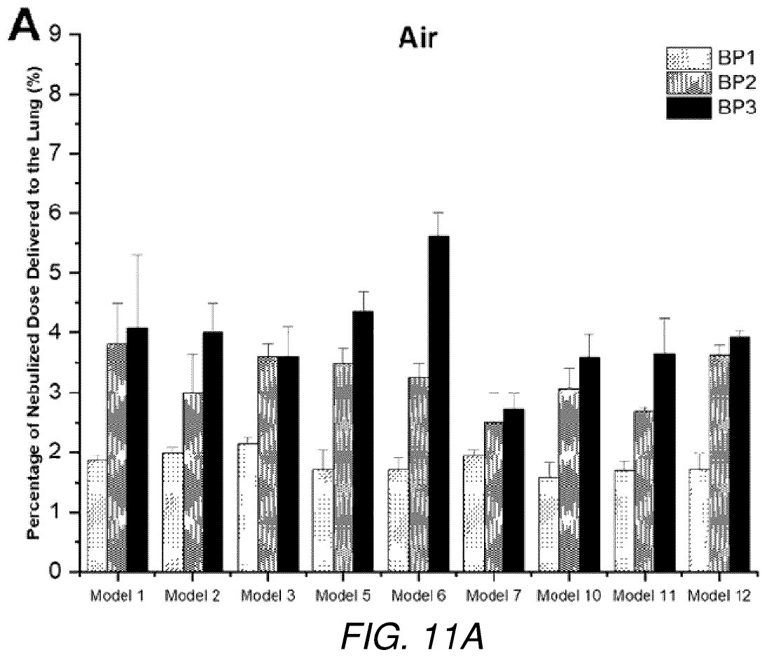

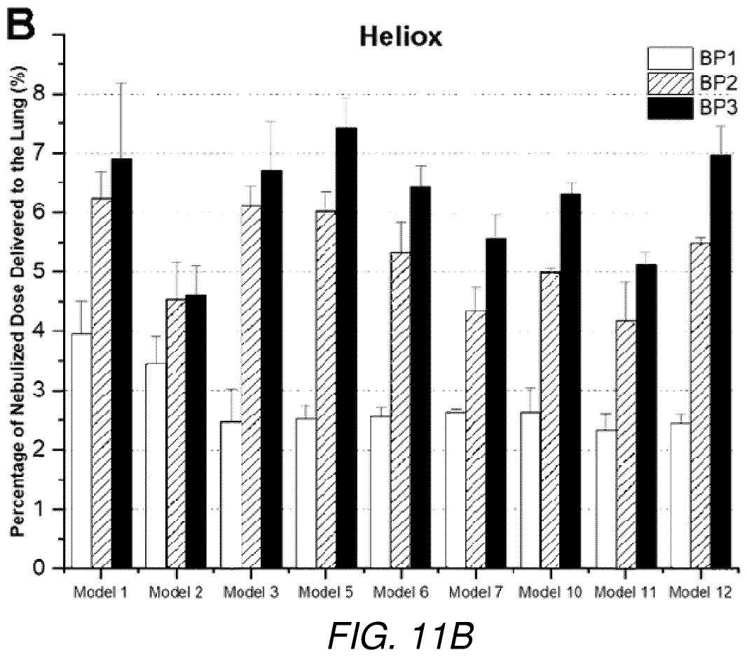

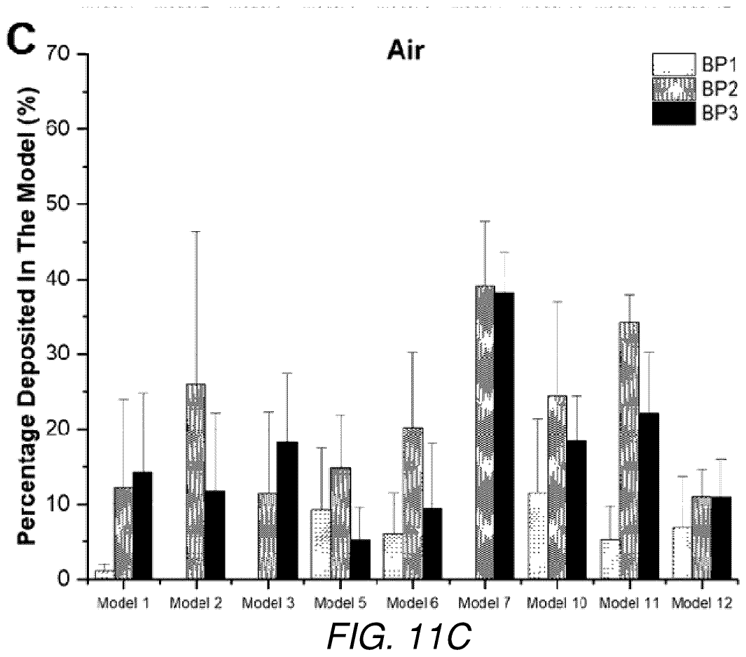

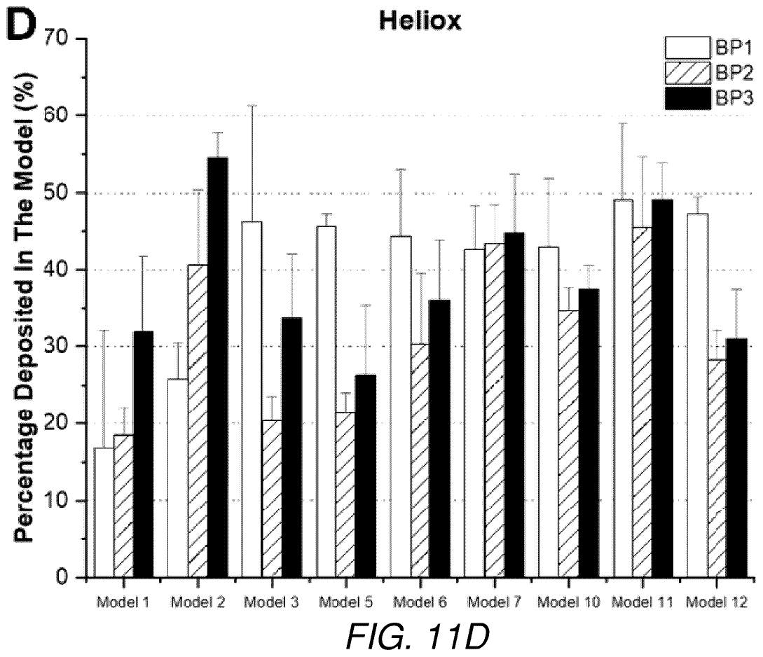

FIGS. 11A and 11B show the average percentage of nebulized dose recovered on the filter under the same conditions, reflecting nebulization efficiency. FIGS. 11C and 11D illustrate aerosol deposition percentages within the airway models for Air (FIG. 11C) and Heliox (FIG. 11D) across the three breathing patterns. Deposition generally increased as inhalation volume rose from BP1 to BP3. Although the absolute amount of drug deposited differs between Air and Heliox, Heliox consistently resulted in a noticeably higher deposition. Overall, these findings emphasize that both inhalation pattern and carrier gas type significantly affect aerosol deposition. Taken together, FIGS. 10A-10D and 11A-11D demonstrate considerable variability in both inhaled drug delivery and nebulization efficiency, with Heliox generally resulting in a higher recovered filter dose.

### Aerosol Sizing

Aerosol size distribution was characterized using an optical particle sizer to determine potential changes in particle size across different regions of each tested model. Measurements included the count median diameter (CD), mass median diameter (2D), and geometric standard deviation (GSD). These parameters were evaluated at the model inlet (representing the inhaled dose) and outlet under each breathing pattern. The resulting CMD, MMD, and GSD values for all models and breathing patterns are summarized in Table 4 for both carrier gases.

TABLE 4
Summary of the aerosol count median diameter (CMD) and mass median diameter (MMD)
and the Geometric Standard Deviation (GSD) entering the models, represented as inhaled,
and at the end of each of the models for both carrier gases and all breathing patterns.
Values are presented as mean (standard deviation) for three replicates (n = 3).

| | | Breathing Pattern 1 | | Breathing Pattern 2 | | Breathing Pattern 3 | |
|---|---|---|---|---|---|---|---|
| | | Air | Heliox | Air | Heliox | Air | Heliox |
| Inhaled | CMD [μm] | 1.94 | 1.82 | 1.56 | 0.89 | 1.25 | 1.17 |
| | | (0.00) | (0.22) | (0.00) | (0.21) | (0.00) | (0.14) |
| | MMD [μm] | 3.01 | 3.01 | 2.52 | 2.82 | 2.24 | 3.75 |
| | | (0.00) | (0.00) | (0.17) | (0.34) | (0.00) | (0.00) |
| | GSD | 1.39 | 1.39 | 1.34 | 1.56 | 1.25 | 1.55 |
| Model 1 | CMD [μm] | 1.94 | 2.26 | 1.17 | 1.38 | 2.15 | 1.25 |
| | | (0.00) | (0.28) | (0.14) | (0.32) | (0.00) | (0.00) |
| | MMD [μm] | 3.01 | 3.75 | 2.13 | 2.42 | 2.42 | 2.42 |
| | | (0.00) | (0.00) | (0.00) | (0.00) | (0.00) | (0.00) |
| | GSD | 1.44 | 1.39 | 1.75 | 2.00 | 1.34 | 1.34 |
| Model 2 | CMD [μm] | 1.94 | 1.94 | 1.17 | 1.01 | 1.01 | 1.27 |
| | | (0.00) | (0.00) | (0.14) | (0.14) | (0.00) | (0.28) |
| | MMD [μm] | 3.01 | 3.01 | 2.13 | 2.53 | 3.01 | 2.42 |
| | | (0.00) | (0.00) | (0.50) | (0.84) | (0.00) | (0.00) |
| | GSD | 1.44 | 1.62 | 1.75 | 1.75 | 1.55 | 1.55 |
| Model 3 | CMD [μm] | 1.94 | 1.94 | 1.25 | 1.01 | 1.01 | 1.27 |
| | | (0.00) | (0.00) | (0.00) | (0.00) | (0.00) | (0.28) |
| | MMD [μm] | 3.01 | 3.01 | 2.13 | 2.53 | 2.42 | 2.42 |
| | | (0.00) | (0.00) | (0.50) | (0.84) | (0.00) | (0.00) |
| | GSD | 1.55 | 1.62 | 1.70 | 1.75 | 1.39 | 1.55 |
| Model 5 | CMD [μm] | 1.94 | 1.94 | 1.25 | 1.25 | 1.01 | 1.19 |
| | | (0.00) | (0.00) | (0.00) | (0.00) | (0.00) | (0.32) |
| | MMD [μm] | 3.01 | 3.75 | 2.13 | 2.53 | 2.42 | 2.82 |
| | | (0.00) | (0.00) | (0.50) | (0.84) | (0.00) | (0.34) |
| | GSD | 1.44 | 1.50 | 1.70 | 1.75 | 1.44 | 1.50 |
| Model 6 | CMD [μm] | 1.94 | 1.63 | 1.46 | 1.38 | 1.36 | 1.17 |
| | | (0.00) | (0.54) | (0.18) | (0.32) | (0.18) | (0.14) |
| | MMD [μm] | 3.01 | 3.75 | 2.13 | 2.53 | 2.42 | 3.01 |
| | | (0.00) | (0.00) | (0.50) | (0.84) | (0.00) | (0.00) |
| | GSD | 1.39 | 1.55 | 1.64 | 1.75 | 1.34 | 1.55 |
| Model 7 | CMD [μm] | 1.82 | 1.94 | 1.36 | 1.82 | 1.09 | 1.17 |
| | | (0.22) | (0.00) | (0.18) | (0.22) | (0.14) | (0.14) |
| | MMD [μm] | 3.01 | 3.75 | 2.13 | 2.53 | 2.82 | 2.82 |
| | | (0.00) | (0.00) | (0.50) | (0.84) | (0.34) | (0.34) |
| | GSD | 1.39 | 1.44 | 1.64 | 1.64 | 1.44 | 1.39 |
| Model 10 | CMD [μm] | 1.56 | 1.56 | 1.25 | 1.09 | 1.17 | 0.94 |
| | | (0.00) | (0.00) | (0.00) | (0.14) | (0.14) | (0.11) |
| | MMD [μm] | 2.82 | 3.26 | 2.13 | 2.53 | 2.42 | 3.01 |
| | | (0.34) | (0.43) | (0.50) | (0.84) | (0.00) | (0.00) |
| | GSD | 1.44 | 1.50 | 1.75 | 1.75 | 1.39 | 1.55 |
| Model 11 | CMD [μm] | 1.56 | 1.94 | 1.01 | 1.17 | 1.01 | 1.09 |
| | | (0.00) | (0.00) | (0.00) | (0.14) | (0.00) | (0.14) |
| | MMD [μm] | 2.82 | 3.75 | 2.13 | 2.13 | 2.42 | 3.01 |
| | | (0.34) | (0.00) | (0.50) | (0.50) | (0.00) | (0.00) |
| | GSD | 1.39 | 1.44 | 1.64 | 1.75 | 1.39 | 1.55 |
| Model 12 | CMD [μm] | 1.56 | 1.01 | 1.25 | 1.19 | 1.25 | 1.01 |
| | | (0.00) | (0.00) | (0.00) | (0.32) | (0.00) | (0.00) |
| | MMD [μm] | 2.82 | 3.75 | 2.42 | 2.82 | 2.42 | 3.26 |
| | | (0.34) | (0.00) | (0.00) | (0.34) | (0.00) | (0.43) |
| | GSD | 1.39 | 1.55 | 1.29 | 1.55 | 1.34 | 1.45 |

### Temperature Considerations

A temperature drop of approximately 8.3° C. (15.0° F.) was observed across the model during benchtop testing with the carrier gas being at 37° C. To maintain isothermal conditions and mitigate cooling effects, three models (Models 1, 6, and 11), representing maximum, intermediate, and minimum aerosol deposition for Air and Heliox with BP2, were retested in an environmental chamber. The inhaled dose was not re-evaluated as temperature decrease from aerosol generation to mouthpiece was relatively small (2.8° C., 5.0° F.). The resulting CMD and MMD for the three models for the heated conditions are presented alongside the benchtop conditions in Table 5.

TABLE 5
Summary of the aerosol count median diameter (CMD) and mass
median diameter (MMD) and the Geometric Standard Deviation
(GSD) at the end of each of the models for both Air and Heliox
for the Breathing Pattern 2. Values are presented as mean
(standard deviation) for three replicates (n = 3).

| | | Unheated | | Heated | |
|---|---|---|---|---|---|
| Breathing Pattern 2 | | Air | Heliox | Air | Heliox |
| Model 1 | CMD [μm] | 1.94 | 2.26 | 0.43 | 0.85 |
| | | (0.00) | (0.28) | (0.09) | (0.28) |
| | MMD [μm] | 3.01 | 3.75 | 2.82 | 2.82 |
| | | (0.00) | (0.00) | (0.34) | (0.34) |
| | GSD | 1.44 | 1.39) | 1.39 | 1.55 |
| Model 6 | CMD [μm] | 1.94 | 1.63 | 0.49 | 0.85 |
| | | (0.00) | (0.54) | (0.06) | (0.28) |
| | MMD [μm] | 3.01 | 3.75 | 4.06 | 2.42 |
| | | (0.00) | (0.00) | (0.53) | (0.00) |
| | GSD | 1.39 | 1.55 | 1.74 | 1.39 |
| Model 11 | CMD [μm] | 1.56 | 1.94 | 0.42 | 0.85 |
| | | (0.00) | (0.00) | (0.00) | (0.28) |
| | MMD [μm] | 2.82 | 3.75 | 2.62 | 3.26 |
| | | (0.34) | (0.00) | (0.34) | (0.43) |
| | GSD | 1.39 | 1.44 | 1.39 | 1.45 |

Generally, CID decreased under heated conditions, except for Heliox in Model 6, which slightly increased. Similarly, MMD decreased or remained consistent except for Air in Model 6. Notably, the bench-top tests showed increases in CMD and MMD when switching from Air to Heliox, whereas under heated conditions, CMD decreased for Air in Models 1 and 11. However, despite a smaller CMD with Air, MMD either increased or remained comparable to Heliox across all models.

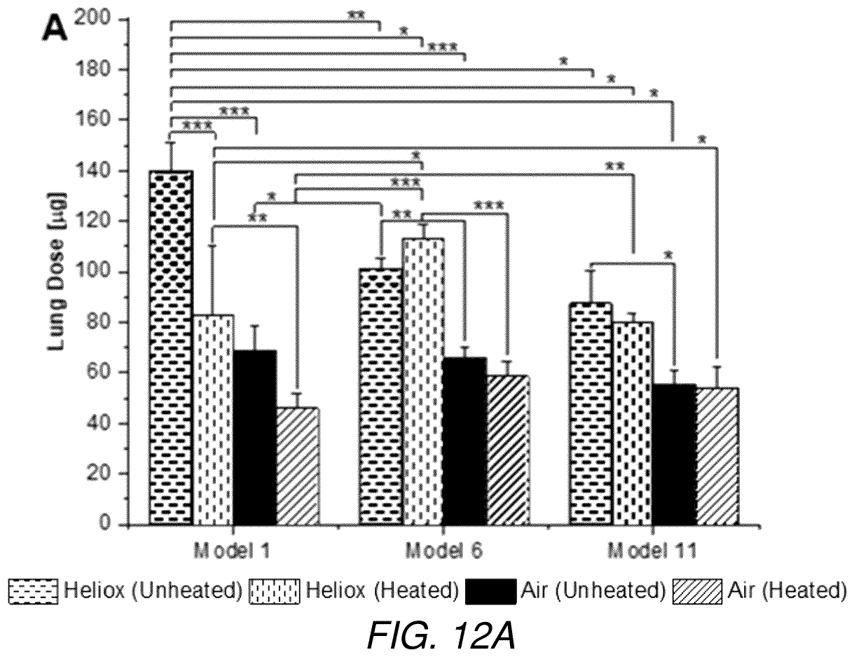

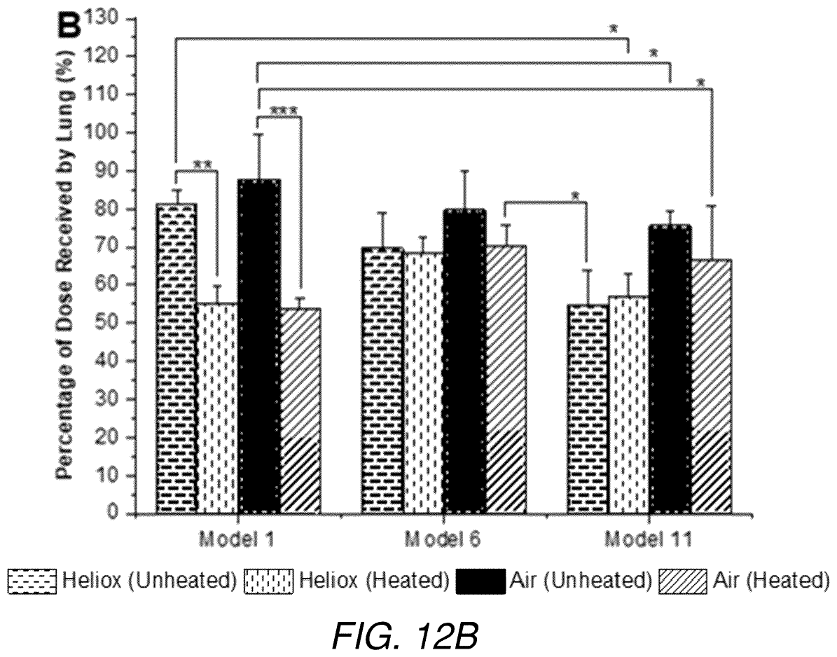

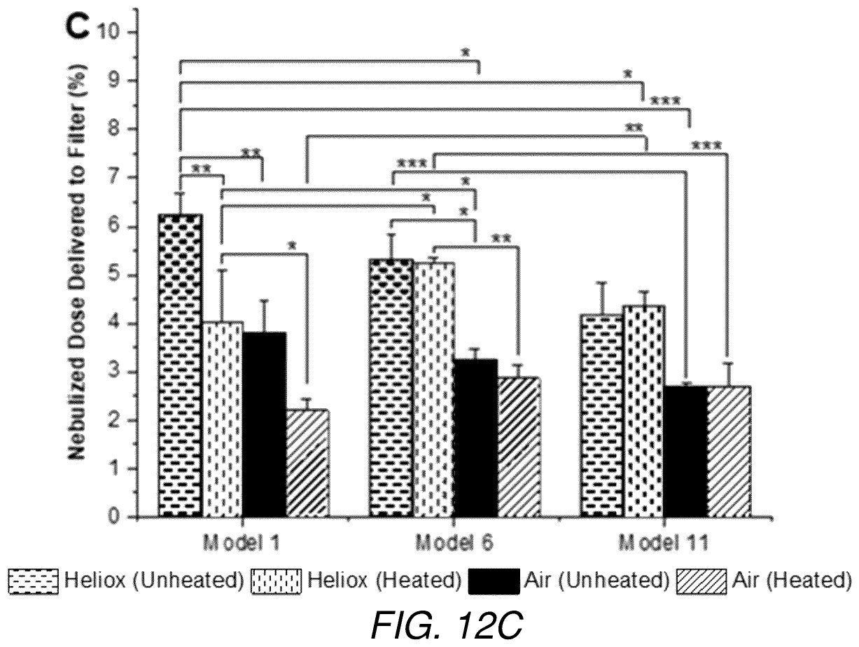

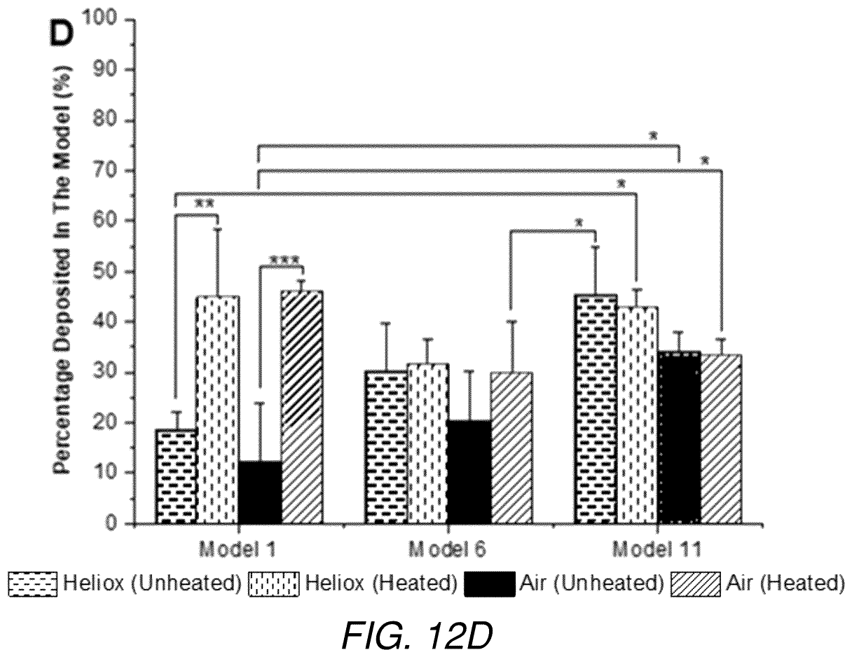

FIGS. 12A-12D compares aerosol deposition from BP2 for both gases in selected models, highlighting temperature and humidity impacts on Lung Dose (FIGS. 12A and 12B). Heating the carrier gas produced variable deposition effects depending on model and gas type; lung dose and the percentage of dose received by lung generally decreased for all the models when heating Air. However, in the case of Heliox, the latter was only observed in Model 1, while Models 6 and Model 11 exhibited minimal changes or slight reductions. These findings indicate temperature and gas properties both significantly influence aerosol behavior, with variations likely tied to airway geometry. FIG. 12C depicts the average nebulized dose percentages recovered at the filter highlighting trends observed previously in FIGS. 11A-11D. FIG. 12D shows the aerosol deposition percentages within the airway models separately for each carrier gas both Heated and Unheated, which trends vary across models and gas types.

For Model 1, heating led to a substantial increase in deposition for both carrier gases. In contrast, Model 6 showed minimal change between conditions, while Model 11 exhibited relatively consistent deposition levels across all scenarios, with only slight variation.

These results suggest that the impact of heating on aerosol deposition is both model-specific and dependent on the carrier gas, highlighting the complex interaction between airway geometry, gas properties, and thermal conditions.

### ANOVA and Pairwise Comparison Outcomes

A two-way ANOVA was performed to assess the effects of carrier gas type and breathing pattern on aerosol deposition. Carrier gas type showed a significant main effect (p<0.0001); in contrast, the overall main effect of breathing pattern was not statistically significant (p<0.2662). However, a significant interaction effect was found between gas type and breathing pattern (p<0.0001), indicating the interplay between the carrier gas's physical properties and specific breathing mechanics. A second ANOVA incorporating carrier gas alongside heating condition, again highlighted the dominant role of gas type. Under unheated conditions deposition with Air was significantly different when the gas was unheated compared to Heliox (p<0.0250).

Pairwise comparisons indicated that switching from Air to Heliox generally produced large, statistically significant changes in deposition, even when breathing pattern varied, supporting the conclusion that gas composition is often the dominant factor. Within a single gas, some breathing pattern comparisons were nonsignificant, suggesting a more subtle effect; however, in certain patterns, clear shifts in deposition emerged, indicating that both gas type and breathing pattern can play meaningful roles. Subsequent multiple comparisons, including Tukey's HSD and Student's t-tests, clarified where these differences were most pronounced.

### Predictive Correlations

In a prior investigation by Golshahi et al. [37], only the Stokes number (Stk), and Reynolds number (Re) were used as non-dimensional parameters for analyzing aerosol deposition in children's oral airway models under constant flow conditions. Specifically, the function n=[1−1/(a*Stk^{b} (Re)^{c}+1)]*100 was employed, which characterizes deposition as a function of relevant non-dimensional parameters: the Stokes number (Stk) and Reynolds number (Re). The study also utilized two distinct characteristic diameters: the ratio of volume to surface area V/Aₛ and the square root of the volume-to-length ratio √(V/L). Here, V represents the airway volume, Aₛ denotes the airway surface area and L represents the airway length.

Universal Fits using V/Aₛ and √(V/L)

Both characteristic diameters: the ratio of volume to surface area V/Aₛ and the square root of the volume-to-length ratio √(V/L) were evaluated for their ability to standardize aerosol deposition predictions across different children oral airways models for both carrier gases. V/Aₛ consistently demonstrated a significant reduction in data scatter, achieving an R² of 0.81, with the corresponding equation to this fit presented in Equation 10 below:

$$PD = a + ( 18.10931 \times b ) + ( 22.93802 \times c ) + ( 124.14589 \times d ) + ( 68.38434 \times e ) + ( - 667.1952997 \times f ) + ( - 347.54158 \times g ) + ( 43.34325 \times h ) + ( - 30.02412 \times i ) + ( - 37.093799 \times j ) + ( - 106.88896 \times k ) + ( - 1640.23805 \times l ) \qquad ( 10 )$$

$$PD = a + ( 0.066071 \times b ) + ( - 10780.02876 \times c ) + ( 188.634076 \times d ) + ( - 0.403149 \times e ) + ( 5.125857 \times f ) + ( 0.022155 \times g ) + ( 6481.655017 \times h ) + ( 53.979029 \times i ) + ( 0.0619098 \times j ) + ( - 0.00013 \times k ) + ( - 27856387.41 \times l ) + ( - 0.6501195 \times m ) \qquad ( 11 )$$

whereas √(V/L) resulted in a slightly lower R² value of 0.74 Equation 11. These results, including estimated coefficients, symbols, standard errors, and p-values, are comprehensively documented in Table 6.

TABLE 6
Predictive Models for the universal fits.

| | | | | Std | |
|---|---|---|---|---|---|
| Model | Term [unit] | Symbol | Estimate | Error | Prob > \|t\| |
| Air | Intercept | a | 43.535338 | 53.54082 | 0.4202 |
| and | Log(Re) | b | 18.109306 | 7.148919 | 0.0146* |
| Heliox | Log(Stk) | c | 22.938018 | 5.835284 | 0.0003* |
| for | (Log(Re) − 8.11159)*(Log(Stk) + 1.5242) | d | 124.14589 | 24.28367 | <.0001* |
| V/Aₛ | Log(Sc) | e | 68.384345 | 20.65273 | 0.0018* |
| | (Log(Re) − 8.11159)*(Log(Sc) + 0.29562) | f | −667.1953 | 127.5035 | <.0001* |
| | (Log(Stk) + 1.5242)*(Log(Sc) + 0.29562) | g | 347.54158 | 69.91291 | <.0001* |
| | (Log(Re) − 8.11159) *(Log(Stk) + | h | 43.343247 | 22.53781 | 0.0604 |
| | 1.5242) *(Log(Sc) + 0.29562) | | | | |
| | Log(TV/V) | i | −30.02412 | 5.770619 | <.0001 |
| | (Log(Stk) + 1.5242)*(Log(TV/V) − 2.31821) | j | −37.0938 | 12.13114 | 0.0036* |
| | (Log(Re) − 8.11159)*(Log(Re) − 8.11159) | k | −106.889 | 21.68479 | <.0001* |
| | (Log(Sc) + 0.29562)*(Log(Sc) + 0.29562) | l | −1640.238 | 440.7686 | 0.0005* |
| Air | Intercept | a | −216.5091 | 53.32292 | 0.0002* |
| and | Re | b | 0.0660708 | 0.022644 | 0.0054* |
| Heliox | Stk | c | −10780.03 | 8110.221 | 0.1902 |
| for | Sc | d | 188.63408 | 55.03142 | 0.0013* |
| \sqrt{V/L} | (Re − 807.356)*(Sc − 0.76521) | e | −0.403149 | 0.265476 | 0.1356 |
| | TV/V | f | 5.1258567 | 2.636059 | 0.0578 |
| | (Re − 807.356)*(TV/V − 11.5716) | g | 0.0221549 | 0.007256 | 0.0037* |
| | (Stk − 0.00162)*(TV/V − 11.5716) | h | 6481.655 | 3516.214 | 0.0716 |
| | (Sc − 0.76521)*(TV/V − 11.5716) | i | 53.979029 | 19.3125 | 0.0075* |
| | (Re − 807.356)*(Sc − | j | 0.0619098 | 0.020498 | 0.0041* |
| | 0.76521)*(TV/V − 11.5716) | | | | |
| | (Re − 807.356)*(Re − 807.356) | k | −0.00013 | 0.00004 | 0.0022* |
| | (Stk − 0.00162)*(Stk − 0.00162) | l | −27856387 | 11600191 | 0.0203* |
| | (TV/V − 11.5716)*(TV/V − 11.5716) | m | −0.65012 | 0.392637 | 0.1044 |

Individual Fits using V/Aₛ and √(V/L)

In addition to universal modeling, separate regression models were developed and tailored specifically to Air and Heliox, separately, evaluating both characteristic diameters independently. For Air, the use of V/Aₛ provided a notably better fit (R²=0.85) compared to √(V/L) (R²=0.74). Similarly, for Heliox, V/Aₛ again showed superior predictive accuracy (R²=0.76) relative to √(V/L), which produced a significantly lower R² value (0.44). Comprehensive details of these best individual fits, including the regression coefficients, standard errors, and associated p-values, are presented in Table 7. The corresponding predictive equations for each of the cases are included in Equations 12 and 13.

$$P D = a + ( 0.0051724 \times b ) + ( 72.55923 \times c ) + ( - 0.157405 \times d ) + ( 1252.0427 \times e ) + ( - 2.08916 \times f ) + ( - 0.0031127 \times g ) + ( - 54.13595 \times h ) + ( 387.054265 \times i ) + ( - 0.963053 \times j ) \qquad ( 12 )$$

$$PD = a + ( 634.70862 \times b ) + ( 10.829301 \times c ) + ( 23.016619 \times d ) + ( 1324.6348 \times e ) + ( 161.37859 \times f ) + ( - 31.80075 \times g ) + ( 79.411249 \times h ) + ( - 91.59631 \times i ) + ( - 136.1818 \times j ) \qquad ( 13 )$$

TABLE 7
Predictive Model for the individual fits utilizing V/Aₛ.

| | | | | Std | |
|---|---|---|---|---|---|
| Model | Term [unit] | Symbol | Estimate | Error | Prob > \|t\| |
| Air | Intercept | a | −739.4235 | 214.6763 | 0.0026* |
| | Re | b | 0.0051723 | 0.001162 | 0.0002* |
| | Stk | c | 72.55923 | 28.25241 | 0.0183* |
| | (Re − 6666.5)*(Stk − 0.21728) | d | 0.1574048 | 0.035677 | 0.0003* |
| | Sc | e | 1252.0427 | 365.6698 | 0.0027* |
| | TV/V | f | −2.089162 | 0.662629 | 0.0050* |
| | Re − 6666.5)*(TV/V − 11.5716) | g | −0.003113 | 0.000536 | <.0001* |
| | (Stk − 0.21728)*(TV/V − 11.5716) | h | −54.13595 | 15.0458 | 0.0018* |
| | (Sc − 0.58664)*(TV/V − 11.5716) | i | 387.05426 | 120.9065 | 0.0045* |
| | (TV/V − 11.5716)*(TV/V − 11.5716) | j | 0.9630525 | 0.191245 | <.0001* |
| Heliox | Intercept | a | −537.5623 | 358.3867 | 0.1492 |
| | Sc | b | 634.70862 | 344.324 | 0.0801 |
| | Log(Re) | c | 10.829301 | 10.01828 | 0.2926 |
| | Log(Stk) | d | 23.016619 | 7.762003 | 0.0077* |
| | (Sc − 0.94377)*(Log(Stk) + 1.41739) | e | 1324.6348 | 716.6145 | 0.0794 |
| | (Log(Re − 7.48458)*(Log(Stk) + 1.41739) | f | 161.37859 | 46.20639 | 0.0023* |
| | Log(TV/V) | g | −31.80075 | 6.864542 | 0.0002* |
| | (Log(Re − 7.48458)*(Log(TV/V) − 2.31821) | h | 79.411249 | 39.76805 | 0.0596 |
| | (Log(Stk) + 1.41739)*(Log(TV/V) − 2.31821) | i | −91.59631 | 30.6439 | 0.0073* |
| | (Log(Re − 7.48458)*(Log(Re) − 7.48458) | j | −136.1818 | 62.44558 | 0.0413* |

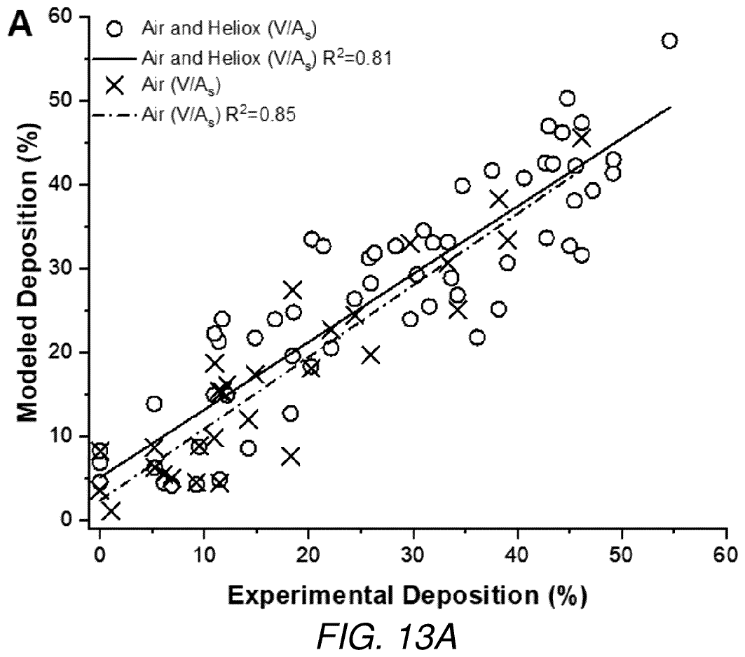

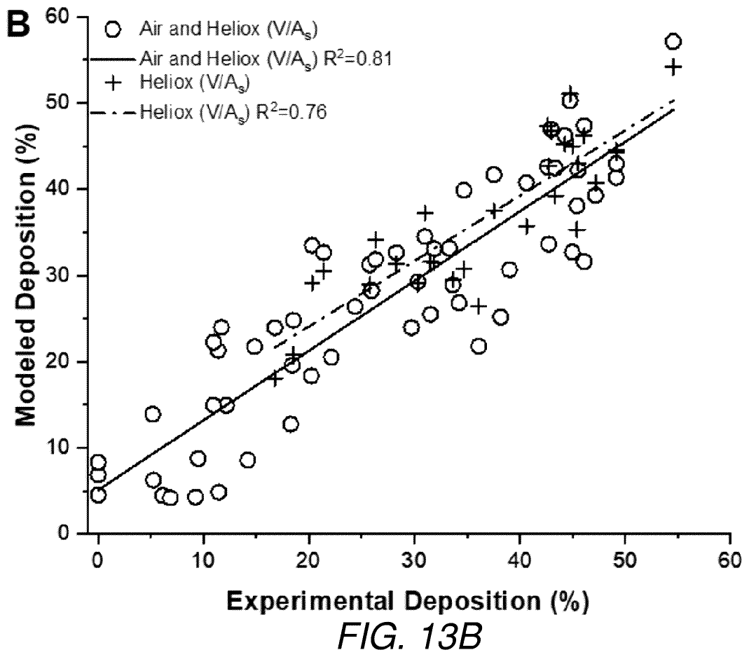

FIGS. 13A and 13B provide graphical comparisons of these regression models. FIG. 13A illustrates both the universal fits and the individual fits specifically for Air, comparing results from both characteristic diameters. Similarly, FIG. 13B provides these comparative results for Heliox. Both panels demonstrate that V/Aₛ consistently reduces data scatter and increases predictive accuracy, reinforcing its greater suitability relative to √(V/L). These observations reinforce the idea that the choice of characteristic diameter can meaningfully impact the accuracy of concentration predictions, particularly when modeling individual gases rather than pooling them into a universal fit.

To further understand the factors driving these predictions, the relative importance of variables within the predictive models was analyzed. While p-values traditionally indicate the statistical significance of individual parameters, a parameter's overall influence can be masked when it is involved in multiple interaction terms. Therefore, we assessed the total effect of each main parameter was assessed to establish a true ranking of their importance in both universal and individual predictive models. In the universal fits the ordering was consistent: Re had the largest influence, followed by Sc, then TV/V, with Stk contributing the least.

In the individual, gas specific models (specifically V/Aₛ), the pattern differed by carrier gas. For Air, TV/V was the dominant factor, followed by Re, Stk, and Sc contributing the least. For Heliox, Stk was the leading factor, followed by TV/V, Re, and Sc again the least. Taken together, these results indicate that although Re is most influential when fits are combined, the relative importance of parameters is gas-dependent: TV/V drives deposition in Air, whereas Stk is most influential in Heliox, with Sc consistently the least important across conditions.

### Discussion

This study evaluated aerosol deposition behavior of nebulized hygroscopic aerosols, specifically albuterol sulfate, within pediatric oral airway models under physiologically relevant breathing conditions. The results demonstrate that hygroscopic and volatile characteristics of the aerosol significantly influence deposition patterns due to ongoing evaporation and condensation processes in the respiratory tract. These dynamic size changes introduce a level of variability that is often not captured by traditional deposition models based on stable particle sizes.

We observed that aerosol particles traveling through the warm, humid airways undergo condensational growth, which affects their aerodynamic properties and eventual regional deposition. The analysis of drug mass recovered at the mouthpiece and model outlets, normalized to the inhaled dose, further shows that both carrier gas type and inhalation volume significantly affect pulmonary delivery efficiency. Larger inhalation volumes generally led to higher deposition, emphasizing the key role of residence time in particle transport. Heliox (a lower density carrier gas) produced a greater percentage of drug recovered at the filter than standard air, validating its potential for deeper lung penetration [21].

Despite the favorable performance of Heliox, significant variability in nebulization efficiency and lung dose was still observed across breathing patterns. This highlights the complex interplay between carrier gas properties, aerosol dynamics, and individual airway geometries, especially in pediatric populations. These experimental observations also served as the basis for subsequent development of predictive correlations, which aimed to quantify deposition outcomes using combinations of dimensionless parameters and statistical modeling techniques.

To address this, several mathematical models were evaluated to identify strong predictive relationships between non-dimensional parameters and aerosol deposition. Linear and nonlinear regression approaches were tested, including multiple linear regression, second-order polynomials (with and without cross-terms), power-law transformations, and logarithmic fits. Predictors included dimensionless numbers such as Reynolds (Re), Schimdt (Sc), and Stokes (Stk) numbers, as well as clinically relevant tidal volume ratios (TV/V). Regularization methods such as LASSO were also employed to minimize overfitting and enhance model interpretability.

Despite these efforts, the predictive accuracy of many models remained moderate. Among all techniques tested, Stepwise regression rose as the most statistically valid approach, consistently providing the best balance between fit and simplicity. The inclusion of additional parameters, such as the Schmidt number (Sc) and tidal volume ratios, significantly improved model accuracy compared to traditional two-variable models. These findings highlight the importance of feature selection and highlight the impact of characteristic diameter choice on deposition predictions.

Moreover, developing gas-specific regression models resulted in better predictive performance (for the case of Air) than generalized models, emphasizing the significance of physical properties of carrier gases (e.g., density, viscosity). These results suggest that specific regression models, sensitive to gas composition and aerosol characteristics, are important for accurate prediction of deposition in pediatric airways.

The use of a vibrating mesh nebulizer and humidified carrier gas proved effective in maintaining aerosol stability during delivery, reducing variability due to aerosol drying. When considering alternative carrier gases such as Heliox, dose adjustments become essential to ensure consistent delivery efficiency due to their lower density and altered flow behavior. Although Heliox reduced turbulence and promoted deeper deposition, predictive models required re-tuning to reflect its distinct influence on flow and particle dynamics.

This Example demonstrates the complexity of aerosol deposition in pediatric airways when using hygroscopic, volatile drugs such as nebulized albuterol sulfate. The dynamic processes of evaporation and condensation during aerosol transport play a critical role in determining regional deposition, particularly in the extrathoracic region where droplet size and airflow characteristics are rapidly evolving. The findings revealed that the carrier gas type significantly influences deposition patterns (p<0.0001), with Heliox generally leading to higher recovered drug doses compared to air. While increasing inhalation volume generally enhanced lung dose across different breathing patterns, offering notable improvements in deposition efficiency. A significant interaction effect between gas type and breathing pattern was also observed (p<0.0001), highlighting the complex interplay between carrier gas properties and respiratory mechanics. These effects were quantified using a standardized, normalized approach that minimized variability in nebulizer output.

Through extensive regression analysis, Stepwise regression was identified as the most statistically accurate method for predicting deposition across various breathing and aerosol conditions. The inclusion of additional dimensionless parameters (Sc, TV/V) and gas-specific adjusting significantly improved model accuracy, emphasizing the need for refined, context-aware predictive frameworks.

### Example 3. Evaluation of Existing Correlations

This Example demonstrates limits of existing mathematical models for predicting aerosol deposition for children.

In a prior investigation by Golshahi et al. (L. Golshahi, M. L. Noga, and W. H. Finlay, “Deposition of inhaled micrometer-sized particles in oropharyngeal airway replicas of children at constant flow rates,” J. Aerosol Sci., vol. 49, pp. 21-31, 2012), only the Stokes number (Stk), and Reynolds number (Re) were used as non-dimensional parameters for analyzing aerosol deposition in children's oral airway models under constant flow conditions. Specifically, the function n=[1−1/(a*Stk^{b} (Re)^{c}+1)]*100 was employed, which characterizes deposition as a function of relevant non-dimensional parameters: the Stokes number (Stk) and Reynolds number (Re).

This Example applies the modeling of Golshahi et al. (2012) but with a number of alternative delivery parameters. The previously developed correlation found by Golshahi et al. (2012) for child oral airway deposition is demonstrated in this Example to provide a reasonable estimation for a population for both air and heliox carrier gases; however, the correlation is shown to be unable to predict the deposition accurately on an individual basis. Moreover, the Golshahi et al. (2012) correlations are shown to be of limited applicability for predicting deposition of hygroscopic, volatile drugs such as nebulized albuterol sulfate. The results showed that the existing correlations developed by Dr. Golshahi are limited in predicting the outcome of drug delivery with nebulizers for each individual as opposed to close approximation of the entire population, particularly when changing the aerosol carrier gas phase to the helium oxygen gas mixture (heliox).

### Materials

Nine oral airway replicas were previously developed from CT imaging in Golshahi et al. (2012). Each model was 3D printed in two halves using an Objet24 printer (Stratasys, Israel) with a 0.05 mm resolution. This ensured that the inside of each model was clean and rid of all support material. Each model was then sealed together using an epoxy resin and was checked for leaks by running compressed air through the model while in a water bath. A custom-designed adapter was used to introduce aerosol to the humidified carrier gas from the Vapotherm Precision Flow machines (Vapotherm, MN, USA). This adapter was printed using a clear plastic material (Accura ClearVue, 3DSystmes, Rockhill, SC), with a 0.05 mm resolution.

A breathing simulator (ASL5000, Ingmar Medical, Pittsburgh, PA) was used to create realistic breathing patterns for the child models. The simulated pattern was measured using a low resistance flow meter (SFM 3000, Sensirion, Switzerland). To collect the dose by-passing the oral airways, a low resistance filter (Pulmoguard filter, Quest Medical, North Easton, MA) was attached to the outlet of model in each test. The air and heliox, both heated and humidified, was delivered using their respective Vapotherm units.

Aerosol was delivered to the models using a vibrating mesh nebulizer (Aeroneb Lab, Aerogen, Ireland). The model drug used was a 2 mg/mL albuterol sulphate solution, which is commonly used in inhaler medications for asthma. Nebulization was controlled using a mass dose controller (DSITM Buxco, MN, USA) which was used to control the duty cycle (ratio of nebulized time to total nebulization time) which cycles every six seconds and the total nebulization time.

Analysis of the filter deposition was accomplished using high performance liquid chromatography (HPLC). The HPLC method used in this study is based on the one described in Delvadia et al. with a Hypersil Gold™ PFP HPLC column (5 um pore size, 3 mm diameter, 150 mm length) (Waters Corp. Milford, MA) (Delvadia R R, Longest P W, Hindle M, Byron P R: In Vitro Tests for Aerosol Deposition. III: Effect of Inhaler Insertion Angle on Aerosol Deposition. J. Aerosol Med. Pulm. Drug Deliv. 2012; 26(3):145-156.).

### Aerosol Deposition Measurements

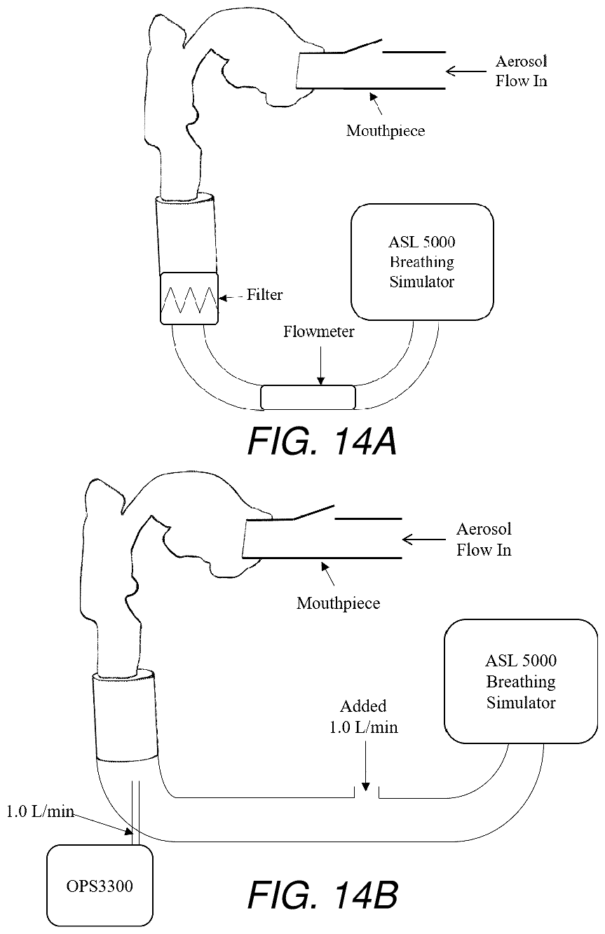

The experimental setup used in this Example for the deposition measurements is shown as a schematic drawing in FIG. 14. A Pulmoguard filter (Quest Medical, North Easton, MA) was attached to the end of the oral airway models and was used to determine the amount of aerosol that was not deposited in the models. A low resistance flow meter (SFM 3000, Sensirion, Switzerland) was used to measure and record the breathing pattern prior to the aerosol delivery. The oral models were attached to a breathing simulator, which was used to generate realistic breathing patterns. A summary of the specific breathing patterns used in this Example are the same provided for Example 2 above, given in Table 3.

### Aerosol Sizing Measurements

The experimental setup used in this Example for the sizing measurements is shown as a schematic drawing in FIG. 14B. Here the Pulmoguard filter used for the deposition measurements has been removed and instead an OPS3300 (TSI, MN, USA) was used to measure the aerosol sizing. The use of an optical sizer, like the OPS3300, allows for heated and humidified carrier gases to be used during the measurements. However, to ensure that the measurements taken were to be accurate for the different carrier gases, dry air and heliox were used to deliver standard sized polystyrene particles, delivered through a jet nebulizer, and subsequently found to have reasonable agreement.

Aerosol sizing measurements were performed at the exit of the custom-designed adapter (prior to the mouthpiece) and at the end of each model. The difference in the sizing profiles determines the relative profile of the aerosol size that was deposited in the model. It should be noted that due to the high concentration of aerosol leaving the custom-designed adapter and the model, the duty cycle of the nebulizer was set to 50% using the Buxco controller during the aerosol sizing measurements. Since the OPS3300 is incapable of counting the aerosol, if the concentration is larger than 3300 counts/mm3, this allowed for the OPS3300 to be able to measure the size distribution over several start and stop intervals. It is assumed that the size distribution of the aerosol was not affected by using the 50% duty cycle.

### Temperature Considerations

Since there will be a temperature difference between the model (room temperature) and the carrier gas flow rate (37 C), temperature will be measured at four locations: 1) at the carrier gas inlet where aerosol is introduced to the system, 2) within the mouthpiece and prior to the inlet of the model, 3) immediately following the filter for the aerosol deposition, and 4) ambient temperature. This was done using four Type K thermocouples to allows us to evaluate the temperature drop across the system to determine the significance of cooling within the system. All of the aforementioned thermocouple placements have been provided in FIG. 14A for visual reference. Additionally, the average temperature measured between the model inlet (Point 2) and the filter outlet (Point 3) were used to determine the properties of the carrier gas (density and viscosity) used for the evaluating non-dimensional correlations.

In order to determine if differences in aerosol deposition exist due to temperature differences between bench-top (room temperature) and body temperature (37 C), models which experienced the highest, middle, and lowest depositions will be tested in an environmental chamber where both the temperature and humidity are controlled. Here the relative humidity of the chamber was set to ~58%, consistent with relative humidity experienced in the local area where bench top experiments were performed and verified using a hygrometer. Temperature in the chamber was set to 37 C to simulate regular body temperature. Both deposition and sizing measurements were evaluated in the environmental chamber utilizing the same experimental setups shown in FIG. 14A and FIG. 14B, respectively.

### High Performance Liquid Chromatography (HPLC) Method

The HPLC method used in this Example is the same HPLC method used and described in Example 2, above.

### Critical Readout

The aerosol deposition measurements from the HPLC and the size measurements are used to determine the predicted deposition from the correlations. Since only the amount of aerosol deposited on the Pulmoguard filter was measured, in order to determine the amount of aerosol deposited in the airways, the following relationship was used:

$${m}_{deposited} = {m}_{entered} - {m}_{filter} = \frac{Δ {m}_{neb} * {C}_{sol}}{{ρ}_{sol}} - {m}_{filter} \qquad ( 14 )$$

where m_{deposited} is the mass deposited in the airway, m_{filter} is the mass of albuterol recovered from the filter and measured via HPLC, and m_{entered} is the inhaled mass which is calculated based on the change in nebulizer mass (Δm_{neb}), the concentration of the nebulized solution (Cₛₒₗ), and the density of the nebulized solution (ρₛₒₗ). Additionally, due to amount of nebulized solution varies per test, the deposition was normalized to the inhaled mass dose. This allows modification of Equation (14) such that

$${m}_{deposited} = {m}_{entered} * ( 1 - \frac{{m}_{filter}}{{m}_{entered}} ) \qquad ( 14 )$$

By normalizing the mass to the entered (“inhaled”) dose this ensures that any differences in the actual nebulized dose is minimized.

Next by using the aerosol sizing measurements, the previously established non-dimensional correlation that was developed using the same models, was used to predict the aerosol deposition in the oral airways using only Stokes (Stk) and Reynolds (Re) numbers, which are defined as

$$Re = \frac{4 {ρ}_{gas} {\overline{Q}}_{inh}}{π {μ}_{gas} {d}_{c}} \qquad ( 15 )$$

$$Stk = \frac{2 {ρ}_{water} {d}_{a}^{2} {C}_{c} {\overline{Q}}_{inh}}{9 π {μ}_{gas} {d}_{c}^{3}} \qquad ( 16 )$$

where ρ_{gas} is the density of the carrier gas (Air at 37° C.: 1.105 kg/m³; 37° C.: 0.377 kg/m³), Q̅ᵢₙₕ is the average flow rate during inhalation, μ_{gas} is the viscosity of the carrier gas (Air at 37° C.: 1.85 E-05 kg/m-s; Heliox at 37° C.: 2.25 E-05 kg/m-s), d_{c} is the characteristic dimension of the airway, ρ_{water} is the density of water (1000 kg/m³), dₐ is the aerodynamic diameter of the particle. Lastly, C_{c} is the Cunningham slip correction factor defined as:

$${C}_{C} = 1 + \frac{λ}{{d}_{p}} [ 2.34 + 1.05 exp ( - 0.39 \frac{{d}_{p}}{λ} ) ] \qquad ( 17 )$$

where λ is the mean free path of the carrier gas (Air at 37 C: 72 nm; Heliox at 37 C: 147 nm). For this Example, d_{c}=V/A_{S}. Based on Golshahi et al. (2012) using the same oral airway models, the airway deposition was predicted using the following equation:

$$η = 1 - \frac{1}{1 + 4 \text{.99} {Stk}^{2.41} {Re}^{- 0.17}} \qquad ( 18 )$$

Using the bin diameters from the OPS3300, along with the average inhalation breathing flow rate the associated with each of the breathing patterns used in this study, Equation 18 was then be used to predict the airway deposition, and subsequently the predicted filter deposition, which was then compared to the mass recovered from the filter and measured using HPLC.

### Statistical Analysis

At least three replicates were used to determine the aerosol size distribution and deposition measurements. Each measurement is represented as the arithmetic mean of the replicates. Student's t-test was used to evaluate if any differences exist between air and heliox regarding nebulizer output. Curve fitting was performed using a nonlinear least square method, with R-squared (R²) being used to evaluate the fit of the curve to the experimental data.

### Challenges and Assumptions

Aerosol deposition was measured based on the aerosol deposition captured on the Pulmoguard filter found at the end of each model. As such the amount of expected model deposition was to be based on the amount of time used for each experiment. A mass dosing controller was used to ensure that each experiment had a forced nebulization time, with the duty cycle of the device was set to 100% to ensure there would be a continuous nebulization for the entire test. However, even with the controller, the mass change of the nebulizer was not completely consistent, varying slightly each time. As such the assumption that the nebulizer was producing aerosol at a consistent rate over the entire nebulization time is not entirely accurate. It is believed that over a long enough nebulization time, that the assumption of relatively consistent nebulization would be accurate; however, this was not verified in this study, and thus is not known if the experiments were preformed long enough for this to not be a factor.

### Aerosol Sizing

An optical particle sizer was used to measure the inhaled aerosol sizing entering the models and the sizing after the models for both air and heliox. The resulting count median diameter (CMD) and mass median diameter (MMD) for each model and inhaled dose are provided in Table 6 for both gasses each breathing pattern.

Heliox was generally found to carry larger particles, compared to air, through all of the models based on the measured CMD and MMD for all of the models. Likewise, BP1 was also found to result in higher CMD and MMD values in the models, likely due to the low average inhalation flow rate experienced. However, there was no discernible trend found between the CMD and MMD measurements during BP2 and BP3 patterns.

TABLE 6
Summary of the aerosol count median diameter (CMD) and mass median diameter (MMD)
entering the models, represented as inhaled, and at the end of each of the models for both air
and heliox and all breathing patterns. Values are presented as mean (standard deviation)
for three replicates.

| | | Breathing Pattern 1 | | Breathing Pattern 2 | | Breathing Pattern 3 | |
|---|---|---|---|---|---|---|---|
| | | Air | Heliox | Air | Heliox | Air | Heliox |
| Inhaled | CMD | 1.8074 | 1.8160 | 1.5670 | 1.6254 | 1.4706 | 1.7056 |
| | | (0.0129) | (0.0026) | (0.0287) | (0.0231) | (0.0405) | (0.0354) |
| | MMD | 2.5507 | 2.9430 | 2.2442 | 2.7768 | 2.1543 | 3.4743 |
| | | (0.0258) | (0.0180) | (0.0321) | (0.0175) | (0.0304) | (0.0585) |
| Model 1 | CMD | 1.8333 | 2.0253 | 1.5941 | 1.6102 | 1.4566 | 1.4351 |
| | | (0.0085) | (0.0351) | (0.0041) | (0.0141) | (0.0166) | (0.0128) |
| | MMD | 2.6455 | 3.0600 | 2.3602 | 2.3660 | 2.3787 | 2.3738 |
| | | (0.0138) | (0.0321) | (0.0165) | (0.0139) | (0.0210) | (0.0523) |
| Model 2 | CMD | 1.7857 | 1.9952 | 1.4937 | 1.5043 | 1.4893 | 1.6305 |
| | | (0.0186) | (0.0368) | (0.0217) | (0.0583) | (0.0314) | (0.5197) |
| | MMD | 2.4747 | 2.1979 | 2.2656 | 2.2647 | 2.7673 | 2.8040 |
| | | (0.0332) | (0.0260) | (0.0243) | (0.0499) | (0.0511) | (1.0694) |
| Model 3 | CMD | 1.8072 | 1.8272 | 1.5649 | 1.5282 | 1.4796 | 1.5493 |
| | | (0.0110) | (0.0171) | (0.0073) | (0.0472) | (0.0273) | (0.0352) |
| | MMD | 2.6698 | 2.9334 | 2.2602 | 2.5375 | 2.2187 | 2.3691 |
| | | (0.0512) | (0.0292) | (0.0335) | (0.0218) | (0.0195) | (0.0104) |
| Model 5 | CMD | 1.8148 | 1.9849 | 1.5999 | 1.5976 | 1.4796 | 1.5754 |
| | | (0.0341) | (0.0468) | (0.0236) | (0.0159) | (0.0223) | (0.0234) |
| | MMD | 2.6168 | 3.2548 | 2.2324 | 2.5922 | 2.2254 | 2.5179 |
| | | (0.0385) | (0.0419) | (0.0288) | (0.1310) | (0.0219) | (0.1754) |
| Model 6 | CMD | 1.8781 | 1.8957 | 1.6515 | 1.6712 | 1.5576 | 1.5846 |
| | | (0.0154) | (0.0300) | (0.0105) | (0.0144) | (0.0226) | (0.0059) |
| | MMD | 2.7693 | 3.1813 | 2.2785 | 2.5011 | 2.1925 | 2.5898 |
| | | (0.0488) | (0.0298) | (0.0404) | (0.0355) | (0.0230) | (0.0941) |
| Model 7 | CMD | 1.7824 | 1.9400 | 1.5994 | 1.7535 | 1.5698 | 1.6506 |
| | | (0.0201) | (0.0182) | (0.0070) | (0.0070) | (0.0279) | (0.0103) |
| | MMD | 2.5456 | 3.1937 | 2.3011 | 2.5466 | 2.4364 | 2.4280 |
| | | (0.0388) | (0.0323) | (0.0181) | (0.0148) | (0.0215) | (0.0110) |
| Model 10 | CMD | 1.7166 | 1.7599 | 1.5875 | 1.6323 | 1.5732 | 1.6232 |
| | | (0.0162) | (0.0105) | (0.0055) | (0.0106) | (0.0073) | (0.0233) |
| | MMD | 2.4543 | 2.9715 | 2.2648 | 2.4868 | 2.3014 | 2.6507 |
| | | (0.0770) | (0.0667) | (0.0159) | (0.0142) | (0.0240) | (0.1072) |
| Model 11 | CMD | 1.7421 | 1.8316 | 1.5811 | 1.6081 | 1.5076 | 1.6250 |
| | | (0.0267) | (0.0052) | (0.0178) | (0.0093) | (0.0046) | (0.0064) |
| | MMD | 2.4561 | 3.0683 | 2.2786 | 2.4145 | 2.2225 | 2.5352 |
| | | (0.0557) | (0.0124) | (0.0327) | (0.0047) | (0.0414) | (0.0166) |
| Model 12 | CMD | 1.7063 | 1.7888 | 1.5983 | 1.6911 | 1.6330 | 1.6648 |
| | | (0.0194) | (0.0057) | (0.0088) | (0.0075) | (0.0217) | (0.0087) |
| | MMD | 2.4711 | 3.3738 | 2.1631 | 2.9669 | 2.2366 | 3.0005 |
| | | (0.0685) | (0.0442) | (0.0302) | (0.0510) | (0.0380) | (0.0358) |

### Aerosol Deposition

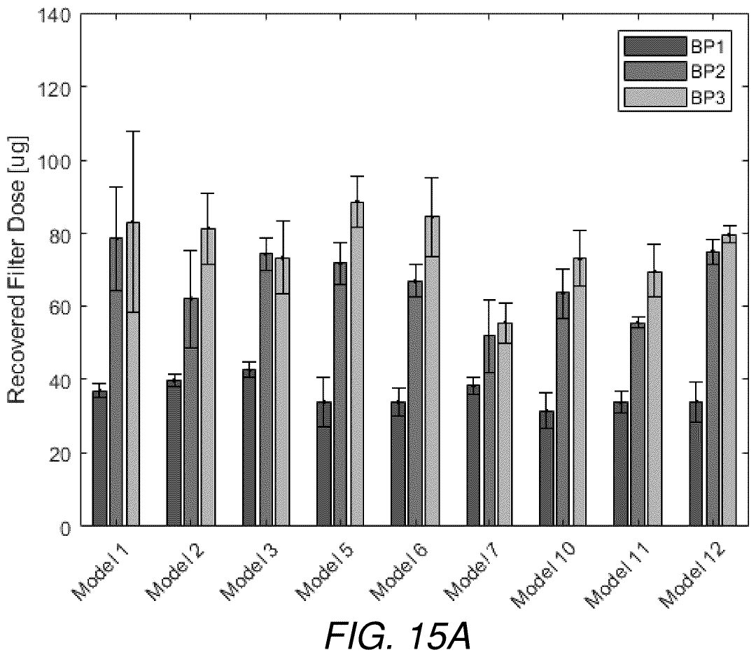

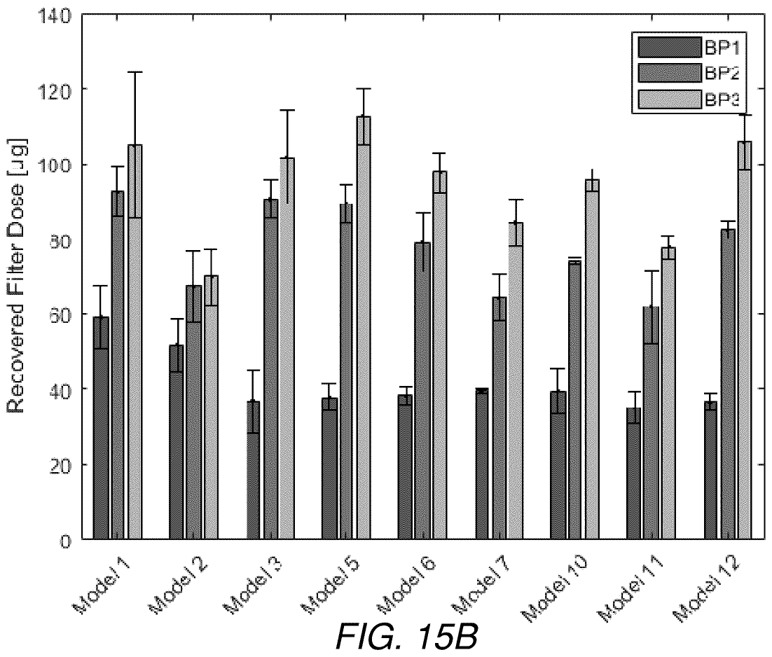

Based on paired Student's t-test there was no significant difference found in the nebulizer output for air and heliox delivery (p=0.85). Aerosol deposition was measured by evaluating the mass of the model drug reaching the filter at either the end of the mouthpiece, to determine the inhaled deposition, or at the end of the model, using high performance liquid chromatography (HPLC). The results for the recovered filter deposition are provided for air and heliox in FIGS. 15A and 15B, respectively.

Here it is clear that the recovered filter dose increased with the breathing pattern for all of the models, likely due to the increase in the total volume inhaled for each pattern. Considering that the total inhaled volume for each pattern was 8.0, 15.4, and 19.4 L for BP1, BP2, and BP3, respectively, while the flow carrying the aerosol remained at a constant 30 LPM, it stands that this would result in a higher recovered dose along the breathing patterns. Additionally, it also shows that heliox generally resulted in a higher recovered filter dose. Considering that CMD and MMD generally increase with heliox for each model, this was also expected. Interestingly though, there was no clear trend that existed among the models across the breathing patterns and carrier gas.

### Temperature Effects

Due to the temperature drop of approximately 15.0 F, or 8.3 C, across the model when testing on the benchtop with the carrier gas being at 37 C, three models were tested in an environmental chamber, where the system could be kept at isothermal conditions. By keeping the system isothermal, this would reduce the effect of cooling that was experienced during the benchtop settings. Based on the results of the aerosol deposition, Models 1, 6, and 11 were selected due to representing the maximum, middle, and minimum deposition among air and heliox for BP2. It should be noted that the inhaled dose was not reevaluated and was considered to be consistent in both testing conditions due to the temperature drop in the setup from aerosol generation to mouthpiece being relatively small (5.0 F or 2.8 C). The resulting CMD and MMD for the three models for the heated conditions are presented alongside the benchtop conditions in Table 7.

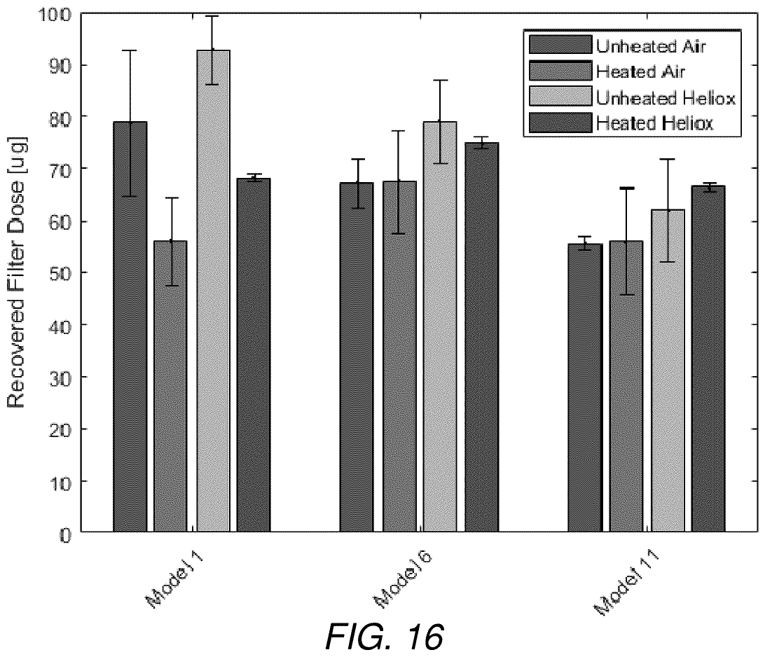

Here the CMD generally decreased in the heated condition, with the exception of heliox for Model 6, which experienced a small increase. The same was experienced for the MMD, generally decreasing in all cases except for heliox in Model 6. Interestingly though, unlike the benchtop case which found an increase in the CMD and MMD when comparing air to heliox, the heated case saw a drop in the CMD in Models 1 and 11. It should be noted though that while the CMD was smaller for heliox in these cases, the MMD increased or remained relatively the same compared to air for all three models. The aerosol deposition was also measured for these three models for the heated condition with the resulting depositions presented alongside the benchtop conditions in FIG. 16.

TABLE 7
Summary of the aerosol count median diameter (CMD) and mass
median diameter (MMD) at the end of each model for the
unheated (benchtop) and heated (environmental chamber)
conditions, for both air and heliox. Values are presented
as mean (standard deviation) for three replicates.

| | | Unheated | | Heated | |
|---|---|---|---|---|---|
| | | Air | Heliox | Air | Heliox |
| Model 1 | CMD | 1.8333 | 2.0253 | 1.7528 | 1.4754 |
| | | (0.0085) | (0.0351) | (0.0130) | (0.1918) |
| | MMD | 2.6455 | 3.0600 | 2.4608 | 2.5295 |
| | | (0.0138) | (0.0321) | (0.0351) | (0.1147) |
| Model 6 | CMD | 1.8781 | 1.8957 | 1.7511 | 1.9207 |
| | | (0.0154) | (0.0300) | (0.0386) | (0.0087) |
| | MMD | 2.7693 | 3.1813 | 2.4153 | 3.7729 |
| | | (0.0488) | (0.0298) | (0.0030) | (0.0687) |
| Model 11 | CMD | 1.7421 | 1.8316 | 1.7388 | 1.5127 |
| | | (0.0267) | (0.0052) | (0.0403) | (0.1388) |
| | MMD | 2.4561 | 3.0683 | 2.4163 | 2.4145 |
| | | (0.0557) | (0.0124) | (0.0725) | (0.0424) |

Here it was found that the recovered filter dose generally dropped or remained the same for all cases, with the exception of heliox in Model 11, which experienced a small increase in the delivered dose. However, only Model 1 experienced a significant drop in the filter mass decreasing 28.4% and 31.5% for air and heliox respectively. Considering the difference in the CMD and MMD between the heated and unheated cases for this model this was expected. Considering that this model did experience the largest filter deposition in the unheated case, this change in the filter deposition would be more significant and potentially explain why the same was not experienced in Model 11, which experienced the lowest filter deposition.

### Evaluating the Previously Developed Correlation

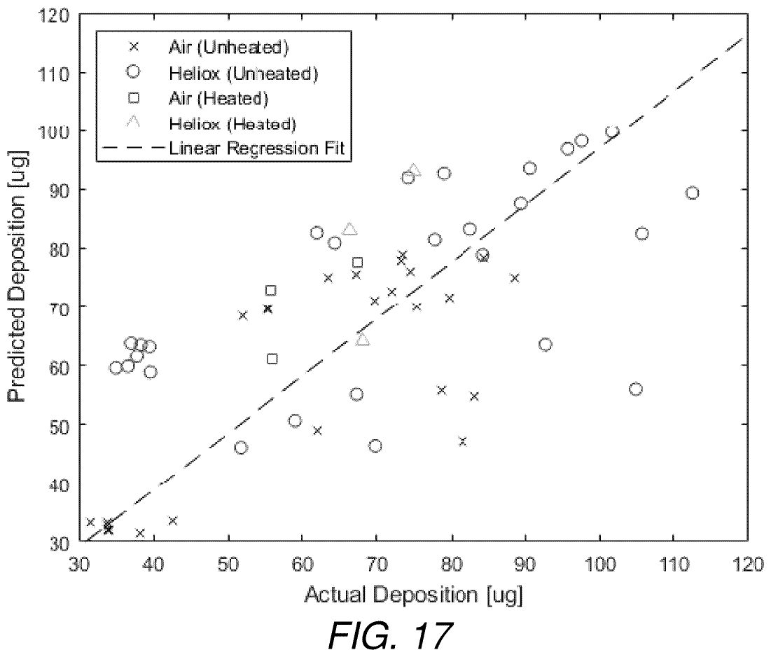

Golshahi et al. (2012) previously developed a correlation to predict the oral airway deposition using the same models in this Example, using Reynolds' (Re) and Stoke's (Stk) numbers. However, this correlation remained untested to carrier gases other than air as well as variances in temperature. Thus, in order to determine if the correlation was able to accurately capture the deposition, the actual measured deposition was compared to the predicted correlation and shown in FIG. 17.

The general trend between the actual and predicted measurements for all the measurements was almost a one-to-one ratio based on a simple linear regression model, with the intercept set to zero, finding the slope of the regression fit to be 0.9705 [95% CI: 0.907, 1.031]. However, despite providing an accurate fit for the population of models, the linear regression also found the fit to have an R² value of 0.3307, implying that the fit is not accurate in capturing individual data points. It is believed that this error results from the heliox data as a regression model of only the unheated air measurements results in an R² value of 0.6240. Meanwhile a regression model of the unheated heliox data was not possible due to resulting in a negative R² value, implying that linear regression fit is worse than fitting the mean of the data. Attempting to develop a new predictive based on the CMD and MMD for each of the models, yielded poor results as no trend was found between the median diameters and the delivered mass.

### Mass Deposition

Evaluation of the deposition data showed that the deposition was significantly affected by changes in the breathing pattern and the carrier gas. Even though it would be expected that the aerosol impaction would increase due to the increase in the flow rates, since the aerosol was added to a constant 30 LPM of flow from the Vapotherm, the deposition was affected by the total inhale volume. This also explains the trend for CMD generally being higher for breathing pattern with the lowest average flow rate, implying that larger particles are passing through the model compared to the other breathing patterns. Meanwhile the increased deposition due to the changing the carrier gas is explained by evaluating the CMD and MMD increases that were experienced between the gases.

### Correlation

Evaluating the deposition correlation developed in Golshahi et al. (2012) showed that while the general trend of the deposition was captured, the correlation was unable to accurately capture individual measurements. Considering that the original correlation was developed in a benchtop setting and only for air, the correlation was able to predict the filter deposition fairly well under these conditions and even produced reasonable results when evaluating the heated conditions. However, the correlation was not able to predict the heliox deposition, even at the lowest flow rate breathing pattern where the deposition was expected to be minimal, the correlation greatly overestimated the heliox deposition for this pattern.

It is believed that differences between the actual and predicted depositions was due to geometric dissimilarity. The accuracy of predicting the deposition data using non-dimensional numbers is reduced, since the differences in the geometry are not scalable from one subject to another. As such, due to non-dimensional numbers reliance on geometry being scalable, these correlations would not be accurate on an individual scale. Additionally, differences in the actual and predicted heliox deposition was also believed to be caused in part to working outside the range of Reynolds numbers for which the correlation was developed, due to the lower density of heliox.

### Temperature Considerations

An environmental chamber was used to evaluate the system under isothermal conditions to eliminate any cooling effect experienced in the benchtop tests. While there was a change in the size distribution of the aerosol based on the changes in the CMD and MMD of the models, the deposition on the filter did not significantly change, except for in one of the models. As such, it is believed that for most of the models, the same deposition measured in the benchtop experiments would also be realized in the environmental chamber.

It is not clear if the changes in the CMD and MMD experienced in the models were due to changes in the aerosol size from potential hygroscopic effects of the model drug (albuterol sulphate) or due to cooling in the benchtop studies. However, even though albuterol sulphate does exhibit hygroscopic characteristics, the same effects would be present in clinical settings. Additionally, by measuring the aerosol sizing at the end of the models, any hygroscopic growth that would be experienced while traveling through the model, would be captured.

### CONCLUSION

This Example utilized oral airway models developed by Golshahi et al. (2012) to evaluate the airway deposition when using alternative carrier gasses. The CMD and MMD for each model increased when heliox was the carrier gas compared to air. As such, the deposition on the filter and the end of the models also increased with heliox. The previously developed correlation found by Golshahi et al. (2012) for child oral airway deposition was found to provide a reasonable estimation for a population for both air and heliox; however, the correlation was found to be unable to predict the deposition accurately on an individual basis. A linear regression fit of all the data resulted in a R² value of 0.331, with the fit being more accurate for the air data with an R² value of 0.624. Meanwhile a regression fit of the heliox data resulted in the fit being worse than fitting the mean of the data. Additionally, there was no discernable trend that found between the measured CMD and MMD for each model and the filter deposition for both air and heliox.

Some embodiments of the present invention may be a system, a device, a method, and/or a computer program product. A system, device, or computer program product may include a computer readable storage medium (or media) having computer readable program instructions thereon for causing a processor to carry out aspects of the present invention, e.g., processes or parts of processes or a combination of processes described herein.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Processes described herein, or steps thereof, may be embodied in computer readable program instructions which may be paired with or downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, or either source code or object code written in any combination of one or more programming languages, including an object oriented programming language such as Java, Python, Smalltalk, C++ or the like, and conventional procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions and in various combinations.

These computer readable program instructions may be provided to one or more processors of one or more general purpose computers, special purpose computers, or other programmable data processing apparatuses to produce a machine or system, such that the instructions, which execute via the processor(s) of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

Where a range of values is provided in this disclosure, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, representative illustrative methods and materials are described.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

As will be apparent to those of skill in the art upon reading this disclosure, each of the individual embodiments described and illustrated herein has discrete components and features which may be separated from or combined with the features of any of the other several embodiments without departing from the scope or spirit of the present invention. Any recited method can be carried out in the order of steps recited or in any other order which is logically possible. Alternative methods may combine different elements of specific detailed methods described above and in the figures.

While exemplary embodiments of the present invention have been disclosed herein, one skilled in the art will recognize that various changes and modifications may be made without departing from the scope of the invention as defined by the appended claims.

1. An airway model, comprising
a plurality of parts each representing a different section of an extrathoracic airway,
wherein the parts are configured to assemble and disassemble,
wherein geometries of the plurality of parts are based on high resolution medical images collected during controlled inhalation of at least one subject.

2. The airway model of claim 1, wherein the high resolution medical images are medical DICOM images.

3. The airway model of claim 2, wherein the DICOM images are computed tomography (CT) images and/or magnetic resonance imaging (MRI) images.

4. The airway model of claim 1, wherein the plurality of parts are configured to permit disassembly after testing to explore regional deposition of pharmaceutical aerosols including one or more of nebulized solutions, suspensions, pressurized metered dose inhaler (pMDI) aerosols, and dry powder inhaler (DPI) aerosols.

5. The airway model of claim 1, further comprising means for maintaining the plurality of parts in an assembled state during use of the airway model.

6. The airway model of claim 1, wherein the plurality of parts comprise
a first section modeling an upper part of an airway; and
a second section modeling a lower part of an airway.

7. The airway model of claim 1, wherein the plurality of parts comprise
a first section modeling the oral cavity;
a second section modeling the oropharynx and larynx; and
a third section modeling the upper trachea.

8. The airway model of claim 1, wherein the airway model is mountable on pharmaceutical aerosol sizing equipment.

9. The airway model of claim 1, wherein the plurality of parts comprise solvent resistant material.

10. The airway model of claim 9, wherein the solvent resistant material is metal.

11. A set of pediatric and/or adult individual mouth-throat airway models for testing combination pharmaceutical aerosol drug delivery products in terms of regional extrathoracic deposition, wherein the models capture anatomical morphologies of mouth-throat geometries, wherein the models capture intersubject variability.

12. The set of claim 11, wherein at least one model of the set comprises
a plurality of parts each representing a different section of an extrathoracic airway,
wherein the parts are configured to assemble and disassemble,
wherein geometries of the plurality of parts are based on high resolution medical images collected during controlled inhalation of at least one subject.

13. The set of claim 12, wherein the high resolution medical images are medical DICOM images.

14. The set of claim 13, wherein the DICOM images are CT and/or MRI.

15. The set of claim 12, wherein the plurality of parts are configured to permit disassembly after testing to explore regional deposition of pharmaceutical aerosols including one or more of nebulized solutions, suspensions, pressurized metered dose inhaler (pMDI) aerosols, and dry powder inhaler (DPI) aerosols.

16. The set of claim 12, wherein the at least one model further comprises means for maintaining the plurality of parts in an assembled state during use of the airway model.

17. The set of claim 12, wherein the plurality of parts comprises
a first section modeling an upper part of an airway; and
a second section modeling a lower part of an airway.

18. The set of claim 12, wherein the plurality of parts comprises
a first section modeling the oral cavity;
a second section modeling the oropharynx and larynx; and
a third section modeling the upper trachea.

19. The set of claim 12, wherein each airway model in the set is mountable on pharmaceutical aerosol sizing equipment.

20. The set of claim 12, wherein the plurality of parts comprise solvent resistant material.

21.-36. (canceled)
